(19) United States
(12) Patent Application Publication
(10) Pub. No.: US 2004/0254094 A1
(43) Pub. Date: Dec. 16, 2004

(54) Suppression of cyclin kinase activity for prevention and treatment of infections

(75) Inventors: Thomas Albrecht, Galveston, TX (US); Laurent Meijer, Roscoff (FR); Priscilla Schaffer, Holliston, MA (US); Luis Schang, Alberta (CA)

Correspondence Address: Mark B. Wilson, FULBRIGHT & JAWORSKI L.L.P., SUITE 2400, 600 CONGRESS AVENUE, AUSTIN, TX, 78701, US

(73) Assignees: The Trustees of University of Pennsylvania and Board of Regents; The University of Texas System

(21) Appl. No.: 09/974,174

(22) Filed: Oct. 10, 2001

Related U.S. Application Data
(60) Provisional application No. 60/304,185, filed on Oct. 10, 2000.

(51) Int. Cl. A61K 38/17

(52) U.S. Cl. 514/2; 514/46; 514/50

(57) ABSTRACT

The present invention relates to methods for use in treating or preventing infections. More particularly, the invention relates to methods for screening for modulators that inhibit cyclin-dependent kinase and the use of these putative inhibitors to control proliferation of a DNA virus that is dependent upon events associated with cell proliferation for replication. The DNA virus includes any of the herpesvirus family, and most particularly human cytomegalovirus.

This application claims priority to U.S. Provisional Application Serial No. 60/304,185, which was filed as regular U.S. patent application Ser. No. 09/685,986 on Oct. 10, 2000, and subsequently converted to a provisional application by petition.

The government owns rights in the present invention pursuant to grant number ES06676 from the National Institute of Health.

### BACKGROUND OF THE INVENTION

1. Field of the Invention

The present invention relates generally to the fields of prophylaxis and treatment of viral, bacterial, and parasitic infections. More particularly, it concerns the use of cyclin-dependent kinase inhibitors for blocking replication of any virus, bacterium, or parasite dependent on CDK activity for proliferation. In some specific embodiments, the invention relates to the use of such inhibitors to block replication of DNA viruses.

2. Description of Related Art

I. Cyclin Dependent Kinases

Among the estimated 1,000 to 2,000 human protein kinases, a family of kinases activated by a family of cyclins, the cyclin-dependent kinases (CDKs), has been extensively studied because of their essential role in the regulation of cell proliferation, of neuronal and thymus functions and of transcription (Morgan, 1997; Meiger et. al., 1997; Vogt et. al., 1998 and Meijer et. al., 2000). The first identified CDK, cdc2, was initially discovered as a gene essential for both G1/S and G2/M transitions in yeast (Nurse et. al., 1981). Following the cloning of the human cdc2 homologue, CDK1, by complementation (Lee et. al., 1987), cdc2 homologues were found to be present in all eukaryotes from plants and unicellular organisms to humans. It was also realized that cdc2 was only the first member of a family of closely related kinases (FIG. 1). Following the initial discovery of cyclin B in sea urchin eggs, it was also shown that cyclin B homologues were present in all eukaryotes, and that, here again, it was the first member of a large family of kinase regulators (FIG. 1).

A. CDKs and Related Kinases: Structure

CDKs are Ser/Thr kinases (about 300 amino acids, molecular weight: 33-40 kDa) which display the eleven subdomains shared by all protein kinases (see the protein kinase resource site: http://www.sdsc.edu/kinases). Nine CDKs and eleven cyclins have been identified in man: the known CDK/cyclin complexes are presented in FIG. 1. The CDKs which associate with cyclin F, G and I have not been identified yet. In addition, there are several “CDK-related kinases” with no identified cyclin partner (FIG. 1). These are easily recognized by their sequence homology to bona fide CDKs and by the presence of a variation of the conserved “PSTAIRE” motif, located in the cyclin-binding domain (sub-domain III) (Meyerson, et. al., 1992). Until their associated cyclin is discovered (if any is associated), these “CDK-related kinases” are named following the sequence of their PSTAIRE motif: PCTAIRE 1-3, PFTAIRE, PITAIRE, KKIALRE, PISSLRE, NKIAMRE and the PITSLRE. To be fully active, CDK/cyclin complexes have to be phosphorylated on the residue corresponding to CDK2 Thr160, located on the T-loop of the kinase. This phosphorylation is carried out by CDK7/cyclin H in association with a third protein, MAT1. The CDK subunit must also be dephosphorylated on Thr14 and Tyr15, two residues located at the border of the ATP-binding pocket.

B. CDKs and Related Kinases: Functions

(i) CDKs and Cell Cycle Control

Progression through the G1, S, G2 and M phases of the cell division cycle is directly controlled by the transient activation of various CDKs (FIG. 2). In early to mid G1, extracellular signals modulate the activation of a first set of CDKs, CDK4 and CDK6 associated with D-type cyclins. CDK4/cyclin D1 and CDK6/cyclin D3 phosphorylate the retinoblastoma protein (pRb) and other members of the pRb family. Phosphorylation inactivates pRb, resulting in the release of the E2F and DP1 transcription factors which, in turn, control the expression of genes whose products are required for the G1/S transition and S phase progression, such as CDK2, cyclin E and cyclin A. The CDK2/cyclin E complex, which is responsible for the G1/S transition, also causes further phosphorylation of pRb allowing the release of an increased amount of transcription factors. During S phase, CDK2/cyclin A phosphorylates different substrates allowing DNA replication and the inactivation of the G1 transcription factors. Around the time of the S/G2 transition, CDK1 associates with cyclin A. Slightly later, CDK1/cyclin B appears and triggers the G2/M transition by phosphorylating a large set of substrates such as the nuclear lamins. Phosphorylation of APC, the “Anaphase Promoting Complex”, by CDK1/cyclin B is required for cyclin B proteolysis, transition to anaphase and completion of mitosis. These successive waves of CDK/cyclin assemblies and activations are tightly regulated by post-translational modifications and intracellular translocations. They are coordinated and dependent on the completion of previous steps, through so-called “checkpoint” controls (Morgan, 1997; Meiger et. al., 1997; Vogt et. al, 1998 and Meijer et. al., 2000).

(ii) CDKs and Transcription

Beside their roles in controlling the cell cycle, some CDKs directly influence transcription. The CDK7/cyclin H/MAT1 complex is a component of the TFIIH complex, a basal transcription factor. TFIIH kinase activity is responsible for phosphorylation of the C-terminal domain of the large subunit of RNA polymerase II (CTD RNA pol II), required for the elongation process.

CDK8 associates with cyclin C and has been found in a multiprotein complex with RNA polymerase II. Like CDK7/cyclin H, CDK8/cyclin C phosphorylates CTD RNA pol II, but on different sites, suggesting a distinct mechanism of transcriptional regulation.

CDK9/cyclin T is a component of the positive transcription elongation factor P-TEFb. It is responsible for the Tat-associated kinase activity involved in the HIV-1 Tat transactivation. It also displays CTD RNA pol II kinase activity.

(iii) CDKs and Neural and Muscular Functions

CDK5 has been purified from bovine brain where it associates with cytoskeletal proteins, such as the tau protein and the neurofilaments NF-H and NF-M. CDK5 activity is important for outgrowth of neurites and neuronal development. CDK5 also plays a crucial role in myogenesis and somites organization in Xenopus embryos and in remodeling tissues. There is a clear involvement of CDK5 in the apoptotic process, as illustrated by a positive correlation between the activity of CDK5 and the number of cells undergoing apoptosis, in both developmental and remodeling tissues.

Another interesting aspect of CDK5 is the nature of its associated regulatory subunits, p35 or p25, a 25 kDa protein derived by proteolytic cleavage from the 35 kDa precursor. Despite their evolutionary distance from cyclins, these proteins function as CDK5 activators in place of the classical cyclins. Nevertheless the predicted structure of p35 shows a similar fold to that of cyclins, which explains the efficient activation of CDK5 and also extends the list of potential activators for CDK-related proteins. It was recently shown that conversion of p35 to p25 leads to constitutive activation of CDK5, and alteration of its cellular localization and substrate specificity (Patrick et. al., 1999). CDK5/p25 expression in cultured primary neurons triggers apoptosis (Patrick et. al., 1999). These findings, as well as the accumulation of p25 (Patrick et. al., 1999) and increased CDK5 activity in Alzheimer's disease patients' brains, indicate that CDK5 activation may be involved in the cytoskeletal abnormalities and neuronal death observed in this neurodegenerative disorder.

Finally CDK5 was recently demonstrated as a downstream element of dopamine signaling (Bibb et. al., 1999). When phosphorylated on Thr34 by PKA, the striatum-specific DARPP-32 protein is an inhibitor of phosphatase 1; when phosphorylated on Thr75 by CDK5/p25, DARPP-32 becomes an inhibitor of PKA. In vivo phosphorylation on this site does not occur in p35−/− tissue (Bibb et. al., 1999).

C CDKs and Apoptosis

In addition to a possible role of CDK5 in neuronal cell death, other enzymes of this family may be involved in apoptosis. The PITSLRE family of CDK-related kinases contains more than 20 isoforms derived from three different genes and alternative splicing. A caspase-dependent proteolytic cleavage in the N-terminal region of some of these isoforms leads to a 50 kDa active kinase involved in apoptosis. It has been recently demonstrated that CDK2, in association with an unidentified protein different from cyclin A or E, is upregulated in thymocytes undergoing apoptosis. This CDK activity is required for induction of apoptosis, providing a very interesting link between cell division and cell death (Guo, et. al., 1990).

II. Viruses

For a virus to multiply, it must first infect a cell. Host ranges of different viruses vary considerably. For example, one virus may have a wide host range, whereas the host range for another virus may be a single cell type of a specific animal. Viruses exhibit an array of strategies for expression of their genes and for the replication of their genomes. Viral genomes can be encoded by either RNA or DNA genomes. These genomes may be single or double stranded. DNA viruses can be classified into the following categories: double-strand DNA viruses replicating in the nucleus (e.g., papovaviruses, papillomaviruses, adenoviruses, herpesviruses); double-strand DNA viruses replicating in the cytoplasm (e.g., poxviruses); single-strand DNA viruses (e.g., parvoviruses) and hepadaviruses containing partially double-stranded circular DNA (e.g., hepatitis B virus).

Many of the cells in adult animals, including humans, are terminally differentiated and have a number of impediments to prevent DNA synthesis. For DNA viruses to replicate their DNA in these differentiated cells, they must overcome these constraints. Some DNA viruses such as papovaviruses induce the cell to enter and traverse the entire cell cycle. The DNA genome of these viruses is replicated in part by cellular enzymes along with cellular DNA. For other viruses such as some human herpesviruses, replication in differentiated cells is accomplished in a different manner. These viruses encode their own enzymes for DNA replication. To accomplish this, viruses such as human cytomegalovirus (HMCV) induce partial traverse of the cell cycle. HCMV activates density-arrested cells to enter the cell cycle and proceed through G1 to a stage at or near the G1/S boundary. This results in substantial increases in the pool of precursors required for DNA synthesis. The abundance of cyclins required for other cell cycle events, as D and A, is not increased, so the cells are unable to replicate their own DNA and complete traverse of the cell cycle. Accordingly, replication of these viruses is dependent upon limited activation of the cell cycle and, particularly, on activation of cyclin E/CDK2.

Herpesviruses are among the most prolific viral causes of disease in humans. They are considered the causal agents of chicken pox and shingles (varicella-zoster virus), mononucleosis (Epstein-Barr virus and human cytomegalovirus), recurrent oral (cold sores) and genital lesions and sporadic meningoencephalitis (herpes simplex viruses), birth defects/mental retardation and mild to life-threatening infections in immunocompromised individuals (human cytomegalovirus), Kaposi's sarcoma (human herpesvirus 8), etc. Herpesviruses of animals are also important infectious agents, producing infections in widely divergent species.

Although the pathogenesis of herpesviruses is incompletely understood, it is widely agreed that the human viruses are all capable of forming lifelong persistent infections of their host. These persistent infections may be reactivated from time-to-time, resulting in clinically apparent disease and opportunities for further dissemination of the virus. More than 90% of the world's population is infected with one or more of the herpesviruses. Because of the extent of infection within the human population and the possibility for reactivated infection, herpesvirus infections intrude in nearly everyone's life. Of the recognized human herpesviruses, human cytomegalovirus (HCMV) and herpes simplex viruses (HSV-1 and HSV-2) produce the highest medical impact.

Over the last two decades, knowledge of the cellular and molecular pathogenesis of herpesviruses has improved greatly. For example, it is now well established that HCMV mitogenically activates the cells that it infects. HCMV evokes a cascade of cellular responses immediately after infection that resemble those induced by serum growth factors in serum-arrested cells. These changes include activation of phospholipase C, phospholipase A2, protein kinase C; Ca²⁺ influx; release of cellular Ca²⁺ stores; increased intracellular free Ca²⁺; increased phospholipid and arachidonic acid metabolism; activation of protein kinases; activation of DNA binding proteins and transcriptional activation of a number of cellular genes (Albrecht et. al., 1989 and Albrecht et. al., 1992). This process, in addition to stimulating the cell to enter the cell cycle, stimulates expression of HCMV immediate early proteins. As these viral proteins become available, cyclin E (one of the cellular proteins involved in regulating cell cycle progression) is transcriptionally activated (Bresnahan et. al., 1998), cyclin-dependent kinase 2 (CDK2) is translocated from the cytoplasm to the nucleus (Bresnahan et. al., 1997b), and cellular calpains are activated and mediate the proteolysis of p21cip1 (an inhibitor of CDK2 activity) (Chen et. al., 1998; Albrecht et. al., 1992; and Albrecht et al., 1989). Ultimately, HCMV pushes the cells to a point at or near the G1/S boundary of the cell cycle, where precursors for DNA synthesis are plentiful and the virus can replicate with good efficiency (Albrecht et. al., 1989). Thus, limited cell cycle progression is associated with high yields of infectious virus from HCMV-infected cells.

### SUMMARY OF THE INVENTION

This invention relates to methods of preventing replication of a virus, bacterium, or parasite in an organism comprising administering a cyclin-dependent kinase inhibitor to the organism infected by the virus, bacterium, or parasite.

In specific embodiments, the organism may be an eukcaryote. In particular, the eukaryote may be a mammal. Particularly, the mammal may be a human. Other examples of mammals include, but are not limited to, mice, rats, dogs, cats, guinea pigs, rabbits or monkeys.

The cyclin-dependent kinase inhibitor may be administered to the organism via several different routes. For example, the inhibitor may be administered via a parenteral route. Exemplary parenteral routes include, but are not limited to, intravenous, intramuscular, subcutaneous, intraperitoneal, intra-arterial, intrathecal or transdermal. The inhibitor may also be administered via an alimentary route, e.g., oral, rectal, sublingual or buccal. Also contemplated in the present invention is administering the inhibitor topically or by inhalation.

Another specific embodiment of the present invention also includes a method of treating an organism infected or suspected of being infected by a virus, baterium, or parasite comprising administering a cyclin-dependent kinase inhibitor to the organism. The inhibitor may be administered in a therapeutically effective amount to inhibit replication. The therapeutically effective amount can be from about 0.1 μg/kg to about 1000 μg/kg. Also contemplated is that the inhibitor may be administered in a prophylactically effective amount to inhibit replication. The prophylactically effective amount can be from about 0.1 μg/kg to about 1000 μg/kg.

In a further embodiment, a second agent may be administered to the organism, in conjunction with the cyclin-dependent kinase inhibitors. The cyclin-dependent kinase inhibitor and the second agent may be administered sequentially or simultaneously. In some cases, the second agent may be a traditional or non-traditional antiviral agent. Traditional antiviral agents include, but are not limited to, aciclovir, ganciclovir, famciclovir, cidofovir, vidarabine, idoxuridine, foscarnet, triflyorothymidine, vidarabine, DHPG (9-(1,3-dihydroxy-2-propoxymethyl)guanine), AZT (3′-axido-3′ deoxythymidine), lamivudine or phosphonoacetic acid. Non-traditional antiviral agents may include antineoplastic agents or other compounds that have minimal inhibitor activity but exhibit low toxicity.

Those of ordinary skill in the art will be able to employ readily available resources and obtain comprehensive information regarding anti-viral, anti-bacterial, and anti-parasitic agents that may be used in the context ot the invention. Such information will include dosage regimes and dosage amounts for many such agents. However, those of skill will also be able to modify the assay methods taught herein with regard to cyclin-dependent kinase inhibitors and determine appropriate dosage ranges and regimes for such agents, even if they are not published. Further, those of skill will recognize that, with combination therapies such as those taught herein, it is often possible to obtain an additive, or even synergistic, effect between the cyclin-dependent kinase inhibitor and the second agent. Therefore, those of skill will recognize that it is possible, and perhaps beneficial to modify the dosages of each agent in the combination therapy regimes taught herein from those taught in the art for administration of each agent separately. Of course, the invention also contemplates that a combination of three, four, five, six, seven, eight, nine, ten, or more agents, of which at least one is a cyclin-dependent kinase inhibitor, may be used in the context of the invention.

The present invention also provides methods of screening for a modulator of cyclin-dependent kinase comprising: obtaining a cyclin-dependent kinase; contacting the cyclin-dependent kinase with a candidate substance; and assaying for cyclin-dependent kinase activity. Exemplary cyclin-dependent kinases may include, but are not limited to, CDK1, CDK2, CDK3, CDK4, CDK5, CDK6, CD7, CDK8 or CDK9.

In specific embodiments of the present invention, the candidate substance may inhibit the cyclin-dependent kinase by competitively inhibiting ATP binding. It is contemplated that the candidate substance may be a small molecule, a protein or fragment thereof, or a nucleic acid molecule, specifically a nucleoside analog. For example, the candidate substance may be 6-dimethylaminopurine, isopentenyladenine, olomoucine, roscovitine, CVT-313, purvalanol A&B, flavopiridol, suramin, 9-hydroxyellipticine, toyocamycin, staurosporine, γ-butyrolactone, CGP60474, kenpaullone, alsterpaullone, indirubin-3′-monoxime or hymenialdisine.

In further embodiments, cyclin-dependent kinase activity may be assayed using molecular biology techniques. Such techniques may include, but are not limited to, RNA hybridization, PCR, RT-PCR or immunodetection, e.g., Western blot, ELISA or indirect immunofluorescence.

In yet another specific embodiment, cyclin-dependent kinase protein may be obtained by procuring an expressed cyclin-dependent kinase protein. The cyclin-dependent kinase protein may be isolated from a transgenic or a non-transgenic cell. The transgenic cell may comprise a recombinant nucleic acid sequence encoding the cyclin-dependent kinase protein. The cell (transgenic or non-transgenic) may be a eukaryotic cell or a prokaryotic cell.

A specific embodiment of the present invention may include contacting the cyclin-dependent kinase protein with the candidate substance. Contacting may be performed in cells or a cell free system. Further, contacting the cyclin-dependent kinase protein with the candidate substance may also be performed in vivo.

A further embodiment also provides a method of modifying the candidate substance to enhance the inhibition of cyclin-dependent kinase activity. Modifying the candidate substance may comprise modification of the amino acid or nucleic acid sequence of the candidate substance. Exemplary modifications to the amino acid sequence of the candidate substance may include, but are not limited to, chemical mutagenesis, radiation mutagenesis, truncation of amino acids or point mutation of amino acids. Further, the nucleic acid sequence of the candidate substance may be modified by chemical mutagenesis, radiation mutagenesis, insertional mutagenesis, in vitro scanning mutagenesis or site-directed mutagenesis. In a specific embodiment, the modified nucleic acid sequence can be inserted into an expression vector, which can be used to transfect cells.

In yet another aspect of the present invention, the candidate substance may be modified to enhance the uptake of the candidate substance into cells. For example, the candidate substance may be packaged into nanocapsules or liposomes, or aggregated to polycationic polymers. These techniques are well known and used in the art to deliver compounds to a cell.

In another embodiment, also provided is a method of screening a candidate substance for cyclin-dependent kinase binding activity comprising: providing a cyclin-dependent kinase protein; contacting the cyclin-dependent kinase protein with the candidate substance; and determining the binding of the candidate substance to the cyclin-dependent kinase protein. The candidate substance may be an inhibitor or enhancer of cyclin-dependent kinase. Yet further, the candidate substance may inhibit ATP binding of cyclin-dependent kinase. Such inhibition of ATP binding may include that the candidate substance can bind to the ATP-binding site of the catalytic subunit of cyclin-dependent kinase. Examples of the candidate substance may include, but are not limited to, 6-dimethylaminopurine, isopentenyladenine, olomoucine, roscovitine, CVT-313, purvalanol A&B, flavopiridol, suramin, 9-hydroxyellipticine, toyocamycin, staurosporine, γ-butyrolactone, CGP60474, kenpaullone, alsterpaullone, indirubin-3′-monoxime or hymenialdisine.

In still another embodiment, also provided is a method of screening putative inhibitors of viral, bacterial, and/or parasitic replication comprising: contacting a cell with a virus, bacterium, or parasite; contacting the cell with an inhibitor of cyclin-dependent kinase; measuring a cellular response; and measuring the yield of infectious virus, bacteria, or parasite, if any. One skilled in the art will recognize that measuring the yield of infectious virus, bacteria, or parasite may include measuring the yield of virus specific components involved in replication.

In specific embodiments of the present invention, the cellular response may include, but is not limited to, phospholipase C activity, phospholipase A2 activity, phospholipid mobilization and metabolism, protein kinase C activity, Ca²⁺ fluctuations, other ion fluctuations (e.g., Na⁺, K⁺, NaHCO₃), protein kinase activities, cAMP, cGMP, activation of DNA binding proteins, transcription of cellular genes or modification of the cytoskeleton or adhesion apparatus.

As used herein the specification, “a” or “an” may mean one or more. As used herein in the claim(s), when used in conjunction with the word “comprising”, the words, “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Relationship of cyclins to CDKs. This is a schematic representation linking cyclins to their CDKs as found in vivo.

FIG. 2. Cyclin, CDK, CKI regulation of the cell cylce. Shown is a schematic view representing the points of action for mammalian cyclin/CDK complexes during the cell cycle.

FIG. 3. CDK-mediated activation of E2F. Schematic diagram demonstrating CDK-mediated release of E2F from the retinoblastoma protein.

FIG. 4. Inhibition of HCMV replication. Schematically shows some key events in HCMV replication and points of inhibition by the methods of the present invention.

FIG. 5A and FIG. 5B. Cyclin E, CDK2, and cyclin E/CDK2 complexes after serum stimulation of G0-arrested cells. Fibroblasts at 70-80% confluence were synchronized in a quiescent state (G0) by serum starvation for 48 hr and then stimulated by adding fresh EMEM with 20% FBS. (See Table 4) Cells were harvested at intervals, stained with propidium iodine, and the DNA content was determined by flow cytometry. Cell lysates were also prepared at intervals after stimulation, and resolved by SDS-PAGE. The resolved proteins were transferred to nitrocellulose and probed with either cyclin E (CcnE), or CDK2 antibodies (FIG. 5A). The cell lysates were also immunoprecipitated with cyclin E antibody. The precipitates were resolved by SDS-PAGE, followed by immunoblotting with an antibody against CDK2 (CcnE/CDK2 in FIG. 5A). In addition, immunoprecipitates formed with cyclin E antibodies were assayed for the ability to phosphorylate histone H1 (FIG. 5A). FIG. 5B illustrates quantitative data representing the mean of two independent experiments in which the abundance of a protein or a complex or the activity of a kinase is expressed relative to the abundance or activity that prevailed at the time of serum addition (0 hr).

FIG. 6A, FIG. 6B, FIG. 6C and FIG. 6D. Subcellular localization of cyclin E, CDK2, Cip1, and Kip1 after serum stimulation of G0 fibroblasts. Fibroblasts at 70-80% confluence were grown on glass cover slips, synchronized in a quiescent state (G0) by serum starvation for 48 hrs, and stimulated by addition of fresh EMEM with 20% FBS. Cover slips were fixed at different times after stimulation, and stained with either cyclin E (FIG. 6D), CDK2 (FIG. 6A), Cip1 (FIG. 6B), or Kip1 (FIG. 6C) antibodies.

FIG. 7A and FIG. 7B. Subcellular fractionalization of CDK2. Fibroblastic cells were arrested and subsequently stimulated with serum. Cytosolic and nuclear fractions were prepared, resolved by SDS-PAGE, transferred to nitrocellulose membrane, and probed with CDK2 antibody. FIG. 7A shows abundance of CDK2 in both nuclear and cytosolic fractions in quiescent cells (0) and 24 hrs after stimulation. FIG. 7B shows the abundance of CDK2 in the nuclear fractions derived from LU cells 0, 4, 8, 12, 16, and 24 hr times after serum stimulation.

FIG. 8A and FIG. 8B. Cip1 and Kip1 abundance and association with cyclin E after serum stimulation. Cell lysates were prepared at intervals after serum stimulation. These lysates were resolved by SDS-PAGE, transferred to nitrocellulose, and probed with either Cip1 or Kip1 antibodies (FIG. 8A). The lysates were also immunoprecipated with Cip1 or Kip1 antibody; the precipitates were resolved by SDS-PAGE, followed by immunoblotting with cyclin E antibodies to determine the extent of complex formation. In the experiment shown in FIG. 8B, cell lysates were prepared 4 hrs and 16 hrs after serum addition. The lysates were immunoprecipitated two times with a mixture of Cip1 and Kip1 antibodies. Aliquots of the immunodepleted supernatant fractions were resolved by SDS-PAGE, transferred to nitrocellulose membranes, and probed with antibodies against cyclin E or CDK2 (lanes 1 and 2). In the experiment shown in lanes 3 and 4, aliquots of the immunodepleted supernatant fractions were immunoprecipitated with CDK2 antibodies. The resulting immunoprecipitate was resolved by SDS-PAGE, transferred to nitrocellulose membranes, and probed with antibodies against cyclin E of CDK2 (lanes 3 and 4).

FIG. 9. Kinetics of cyclin E/CKI formation and cyclin E kinase activity. The graph illustrates the quantitative abundance of Cip1/cyclin E and Kip1/cyclin E complex formation and cyclin E-dependent histone K1 kinase activity as a function of time after serum stimulation.

FIG. 10A and FIG. 10B. Cell cycle analysis following productive HCMV infection. LU cells were synchronized by serum deprivation and infected with HCMV for 1 hr. Thereafter, the viral inoculum was replaced with spent, serum-free media, with (FIG. 6B) or without phosphonoacetic acid (PAA), an inhibitor of viral DNA replication (FIG. 6A). The cells were harvested and stained with propidium iodine at intervals after infection, and the DNA content was determined by flow cytometric analysis.

FIG. 11A, FIG. 11B and FIG. 11C. Cyclin E, CDK2, abundance and cyclin E kinase activity after HCMV infection of serum-arrested, subconfluent cells. LU cells were growth-arrested by serum deprivation. The cells were then infected with HCMV, mock-infected, or serum-stimulated. Cell lysates were prepared at intervals, and 40 μg of protein from each was resolved by SDS-PAGE. The proteins were transferred to nitrocellulose and probed with antibodies against cyclin E (FIG. 11A) or CDK2 (FIG. 11B). The lane identified as ML contains an aliquot of an extract prepared from asynchronous, mid-log cells. Cyclin E-associated histone H1 kinase activity was also determined (FIG. 11C).

FIG. 12A, FIG. 12B and FIG. 12C. Cip1 and Kip1 abundance after HCMV infection of serum-arrested, subconfluent cells. LU cells were growth-arrested by serum deprivation. The cells were then infected with HCMV, mock-infected, or serum-stimulated. Cell lysates were prepared at intervals, and 40 μg of protein from each was resolved by SDS-PAGE. The proteins were transferred to nitrocellulose and probed with antibodies against Cip1 (FIG. 12A) or Kip1 (FIG. 12B). The lane identified as ML contains an aliquot of an extract prepared from asynchronous, mid-log cells. The data shown in FIG. 12C represent the average of two experiments in which the abundance of cyclin E (CcnE), CDK2, Cip1, Kip1, and cyclin E kinase activity (Kinase) were measured during the first 24 hrs after HCMV infection.

FIG. 13A. FIG. 13B and FIG. 13C. Cyclin E, CDK2, abundance and cyclin E kinase activity after HCMV infection of density-arrested cells. LU cells were growth-arrested by contact inhibition. The cells were then either HCMV-infected, mock-infected, or serum-stimulated. Cell lysates were prepared at intervals after treatment, and 40 μg of each lysate was resolved by SDS-PAGE. The proteins were transferred to nitrocellulose and probed with antibodies against cyclin E (FIG. 13A) or CDK2 (FIG. 13B). Cyclin E-associated histone H1 kinase activity was also determined, and these data are shown in FIG. 13C.

FIG. 14A, FIG. 14B and FIG. 14C. Cip1 and Kip1 abundance after HCMV infection of density-arrested cells. LU cells were growth-arrested by contact inhibition. The cells were then either HCMV-infected, mock-infected, or serum-stimulated. Cell lysates were prepared at intervals after treatment, and 40 μg of each lysate was resolved by SDS-PAGE. The proteins were transferred to nitrocellulose and probed with antibodies against Cip1 (FIG. 14A) or Kip1 (FIG. 14B). FIG. 14C represents the mean of two experiments in which the abundance of cyclin E (CcnE), CDK2, Cip1, Kip1, and cyclin E kinase activity (Kinase) were measured after infection.

FIG. 15. HCMV gene expression and activation of cyclin E-dependent kinase. Density-arrested LU cells were infected with stock HCMV, HCMV that had been UV-irradiated for 30 min, purified HCMV, or virus-free supernatant prepared from the virus stock. After 24 hr, the cells were harvested and assayed for cyclin E-associated kinase activity.

FIG. 16. Rb phosphorylation following serum stimulation and HCMV infection. LU cells were serum-arrested for 48 hrs. Arrested cells were either stimulated with serum or HCMV-infected. Cell lysates were prepared 24 hrs after treatment, and 100 μg of each lysate was resolved by SDS-PAGE. The proteins were transferred to nitrocellulose and probed with antibody against Rb.

FIG. 17A and FIG. 17B. Subcellular localization of CDK2 in serum-arrested cells following serum-stimulation or HCMV-infection. LU cells were growth arrested by serum-deprivation at subconfluent densities for 48 hrs. Cells were then stimulated with 20% FBS or infected with HCMV for 24 hrs. Cells were fixed for immunofluorescence and stained with CDK2 antibody (FIG. 17A). Cytosolic and nuclear fractions were prepared. Aliquots of each fraction were resolved by SDS-PAGE, transferred to nitrocellulose membrane, and probed with CDK2 antibody. The abundance of CDK2 in both nuclear and cytosolic fractions in quiescent cells (0 hrs) and 24 hrs after serum stimulation or HCMV-infection are shown (FIG. 17B).

FIG. 18A and FIG. 18B. Subcellular localization of CDK2 in contact-inhibited cells following serum-stimulation or HCMV-infection. LU cells were growth-arrested by contact-inhibition, then stimulated with 10% FBS or infected with HCMV for 24 hrs. Cells were fixed for immunofluorescence and stained with CDK2 antibody (FIG. 18A). Cytosolic and nuclear fractions were prepared. Aliquots of each fraction were resolved by SDS-PAGE, transferred to nitrocellulose membrane, and probed with CDK2 antibody. The abundance of CDK2 in both nuclear and cytosolic fractions in arrested cells (0 hrs) and 24 hrs after serum-stimulation or HCMV-infection are shown (FIG. 18B).

FIG. 19A and FIG. 19B. CDK2 activation in HCMV-infected cells. LU cells were growth arrested by contact inhibition and infected with HCMV. Prior to infection (0 Hr) or 48 hr post-infection cells were harvested and equal amounts (100 μg) of cell lysates immunoprecipitated with CDK2 antibody. The resulting immunoprecipitates were assayed for kinase activity using either Rb or histone H1 as a substrate (FIG. 19A). Cell lysates were also immnunoprecipitated with either cyclin E or cyclin A antibodies and assayed for kinase activity using histone H1 as a substrate (FIG. 19B).

FIG. 20A, FIG. 20B, FIG. 20C and FIG. 20D. Inhibition of cyclin E/CDK2 activity, HCMV DNA synthesis and virus yields of roscovitine. LU cells were growth arrested by contact inhibition and infected with HCMV. 48 hr post-infection cell lysates were prepared and 100 μg of total protein was immunoprecipitated with cyclin E antibody and in vitro kinase activity determined in the presence of the indicated concentration of roscovitine (FIG. 20A). Cells were also infected and treated with various concentrations of roscovitine following infection. 72 hr post-infection, the cells were harvested, total DNA isolated, and the abundance of HCMV DNA determined by slot blot hybridization using a specific HCMV DNA probe (FIG. 20B). 96 hr post infection, infected cells were lysed by freeze-thaw, followed by sonication. Cellular debris was removed by sedimentation and the HCMV containing supernatants were assayed for infectivity by plaque assay (FIG. 20C). Values represent the average of three independent experiments with standard errors shown. FIG. 20D schematically shows the structure of roscovitine.

FIG. 21A, FIG. 21B and FIG. 21C. Inhibition of HCMV DNA sythesis and virus yields by olomoucine. LU cells were growth arrested by density-arrest. Cells were infected and treated with various concentrations of olomoucine following infection. 72 hr post-infection, the cells were harvested, total DNA isolated, and the abundance of HCMV DNA determined by slot blot hybridization using a specific HCMV DNA probe (FIG. 21A). 96 hr post infection, infected cells were lysed by freeze-thaw, followed by sonication. Cellular debris was removed by sedimentation and the HCMV containing supernatants were assayed for infectivity by plaque assay (FIG. 21B). Values represent the average of three independent experiments with standard errors shown. FIG. 21C schematically shows the structure of olomoucine.

FIG. 22A, FIG. 22B, FIG. 22C and FIG. 22D. Hematoxylin and eosin staining of of HCMV-infected cells treated with roscovitine. LU cells were treated with 15 μM roscovitine for 96 hr and stained with hematoxylin and eosin (FIG. 22A). LU cells were also infected with HCMV and treated with either 0 μM (FIG. 22B), 5 μM (FIG. 22C), or 15 μM (FIG. 22D) roscovitine following infection. Infected cells were harvested 96 hr post-infection and stained with hematoxylin and eosin.

FIG. 23A and FIG. 23B. Effects of roscovitine on non-infected LU cells. LU cells were treated with 15 μM roscovitine for 96 hr. After 96 hr cells were stained with propidium iodine and analyzed by flow cytometry (FIG. 23A). In parallel, roscovitine containing medium was removed and replaced with fresh EMEM containing 10% FBS or EMEM containing 10% FBS and bromodeoxyuridine. Cells were harvested 24 hr after removal of roscovitine and analyzed for cell cycle progression by flow cytometry (FIG. 23A) and bromodeoxyuridine incorporation (FIG. 23B).

FIG. 24. Expression and activity of wild-type and dominant negative CDK2. U-373 cells were transiently transfected with either HA-tagged wild type CDK2 (CDK2-wt-HA) or dominant negative CDK2 (CDK2-dn-HA). Cells were then harvested 48 hr later and assayed for HA expression (Western) and HA-associated kinase activity (H1 Kinase).

FIG. 25A, FIG. 25B, FIG. 25C and FIG. 25D. Inhibition of HCMVlate antigens in cells expressing dominant negative CDK2. U-373 cells were transiently transfected with either HA-tagged wild type CDK2 (CDK2 wild type) or dominant negative CDK2 (CDK2 dominant negative). Cells were then seeded onto glass cover slips and infected with HCMV 24 hr after transfection. The cells were fixed 72 hr post-infection with acetone:methanol (1:1) and dual immunofluorescent staining was done for HCMV UL80.5 (Rhodamine) and HA (FITC) expression. (FIG. 25A and FIG. 25B) HA antigen (CDK2) was detected by fluorescein fluorescence to demonstrate cells expressing either wild type or dominant negative CDK2. (FIG. 25C and FIG. 25D) HCMV UL80.5 late antigens were detected by rhodamine fluorescence. The identical field of cells is shown in FIG. 25A and FIG. 25C. An identical field of cells is also illustrated in FIG. 25B and FIG. 25D.

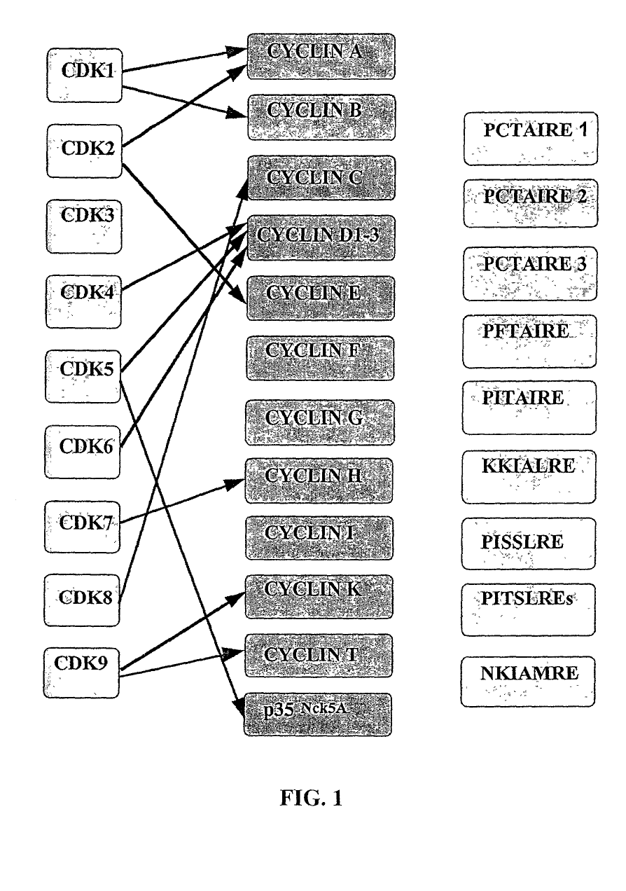

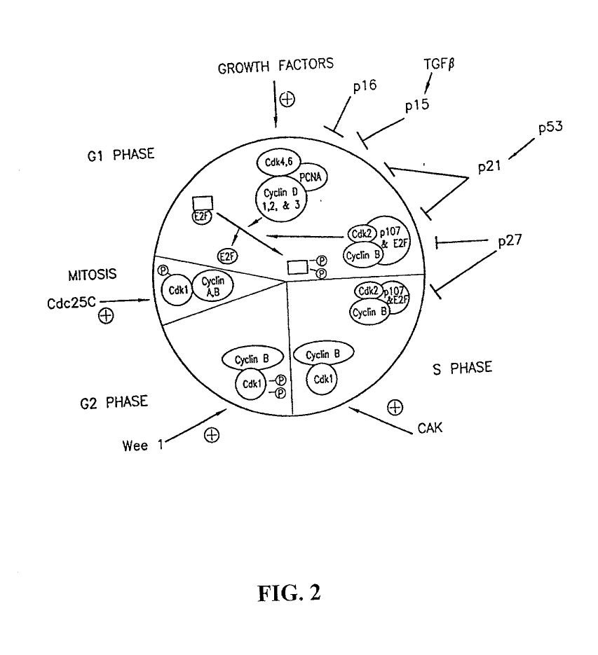

### DESCRIPTION OF ILLUSTRATIVE EMBODIMENTS

There is an urgent need for the development and application of new drugs against viruses, bacteria, and parasites.

For example, DNA viruses, particularly herpesviruses such as cytomegalovirus, particularly human cytomegalovirus (HCMV) and Herpes simplex. These latter viruses are a major problem in modern medicine, especially when the immune system is compromised. When the immune system is compromised, herpesvirus infections become an important cause of morbidity and mortality. Although a number of drugs have recently become available to treat herpesvirus infections, none of these are entirely satisfactory. Most have a level of toxicity that is problematic, particularly with the long-term treatment often required for herpesviruses. Unfortunately, herpesviruses also develop resistance to these drugs surprisingly quickly. Thus, there is not only a need for new drugs, but more importantly a need for new drugs that act through novel mechanisms. This latter point is important because drugs that function through different mechanisms, when combined, often produce a synergistic effect which may or may not be desirable or might even prove deleterious to the patient. Furthermore, resistance to antivirals occurs much less frequently with drugs working through different antiviral mechanisms. At present, antivirals based on inhibition of CDK in particular are not available on the market. Drugs which inhibit CDKs such as CDK2 offer the potential for potent antiviral activity through a novel mechanism. Several inhibitor drugs are described and used herein. Others may be developed by using the screening methods described herein.

I. Pathogenic Organisms

The present invention has applications therefore in the prevention and treatment of viral diseases. Specifically, the present invention may inhibit viral replication or proliferation, particularly viruses that are dependent on CDK activity for proliferation. The following list of viral families that infect humans and animals include, but are not limited to Picornaviridae, Caliciviridae, Astroviridae, Coronaviridae, Paramyxoviridae, Rhabodoviridae, Filoviridae, Orthomyxoviridae, Bunyaviridae, Arenaviridae, Reoviridae, Birnaviridae, Retroviridae, Hepadnaviridae, Ciroviridae, Parvoviridae, Papovaviridae, Adenoviridae, Herpesviridae, Poxviridae, and Iridoviridae. Thus, it is within the scope of the present invention that any specific viral species (known or unknown) contained within one of the above viral families is included in the present invention. Exemplary viral species include, but are not limited to influenza A, B and C, parainfluenza, paramyxoviruses, Newcastle disease virus, rotavirus, respiratory syncytial virus, measles virus, mumps virus, adenoviruses, adenoassociated viruses, parvoviruses, Epstein-Barr virus, rhinoviruses, coxsackieviruses, echoviruses, reoviruses, rhabdoviruses, lymphocytic choriomeningitis virus, coronavirus, polioviruses, herpes simplex, human immunodeficiency viruses, cytomegaloviruses, papillomaviruses, virus B, varicella-zoster, poxviruses, rubella, rabies, picornaviruses, rotavirus and Kaposi associated herpes virus.

In addition to the viral diseases mentioned above, the present invention is also useful in the prevention, inhibition, or treatment of bacterial infections, including, but not limiting to, the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcusfaecalis, Enterococcusfaecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary organisms not in the family Enterobacteriacea include, but are not limited to, Rickettsia, Ehrilichia, Coxiella, Pseudomonas aeruginosa, Stenotrophomonas maltophilia, Burkholderia, Cepacia, Gardenerella, Vaginalis, and Acientobacter species.

In addition, the invention provides methods useful in controlling yeast molds, fungi, protozoan, metazoan or macroscopic infections by organisms. Exemplary yeast molds and fungi include, but are not limited to Candida, Trichosporan, Torulopis, Histoplasma capusulatum, Blastomyces dermatitidis, Paracoccidioides brasiliensis and Coccidioides immitis. Examples of protozoans and metazoans include, but are not limited to Cryptosporidium, Isospora belli, Toxoplasma gondii, Trypanosoma species, Trichomonas vaginalis, Cyclospora species platyhelmithes, nematoda and arthropods. Yet further, the present invention also includes other pathogenic agents, such as prions. Prions are unconventional slow viruses which cause spongiform encephalopathies (slow neurodegenerative diseases). Of course it is understood that the invention may be used on any pathogen against which a composition comprising a CDK inhibitor or a combination of a CDK inhibitor and another agent is effective against the life-cycle of the pathogen.

II. Anti-Viral, Anti-Bacterial, and Anti-Parasitic Drug Development

In the development of safe and effective drugs, it is vital to distinguish the specific functions essential to the virus, bacterium, or parasite that are not present or essential to the cells of the host organism. Stages of viral function that may be vulnerable to antiviral intervention include, but are not limited to, binding to viral receptor, penetrating the cell, pathologic modifications of the cell required for efficient virus replication, mRNA function, DNA synthesis, viral assembly or transport, release of the virus, viral gene regulation, post-translational modification of viral proteins and production of precursors for viral metabolism.

The classes of compounds that are contemplated by the present invention include, but are not limited to, inhibitors of cyclin-dependent kinase activities, i.e., inhibitors of CDK2 activity, nucleoside analogs, cytokines, proteinase inhibitors and non-nucleoside reverse transcriptase inhibitors. Also included in the present invention are other nucleic acid-based antiviral compounds, which could potentially target not only an active virus, but also an inactive virus. Such compounds that are considered may be decoy RNA, antigene, antisense or ribozyme. The use of a decoy involves the production of a short “decoy” RNA from an introduced gene, which corresponds to a regulatory region of the viral genome or transcript. Further, the use of an antigene, instead of antisense, produces DNA which binds to the viral DNA forming a region of a “triple helix”, thus limiting transcription. Methods relating to the production of recombinant DNA, antisense, and ribozymes are discussed elsewhere in this document.

III. Screening for Modulators

The present invention comprises methods for identifying modulators that affect the function of cyclin-dependent kinase protein. These assays may comprise random screening of large libraries of candidate substances; alternatively, the assays may be used to focus on particular classes of compounds selected with an eye towards structural attributes that are believed to make them more likely to modulate the function of cyclin-dependent kinase protein.

By function, it is meant that one may assay for mRNA expression, protein expression, protein activity or binding activity of cyclin-dependent kinase.

A. Modulators and Assay Formats

(i) Assay Formats

The present invention provides methods of screening for modulators of cyclin-dependent kinase activity, e.g., expression of cyclin-dependent kinase proteins or nucleic acids.

In one embodiment, the present invention is directed to a method of:

(a) obtaining a cyclin-dependent kinase protein;

(b) contacting the cyclin-dependent kinase protein with a candidate substance; and

(c) assaying for cyclin-dependent kinase protein activity

wherein a difference between the measured activity indicates that said candidate modulator is, indeed, a modulator of the cyclin-dependent kinase activity.

In yet another embodiment, the assay looks at cyclin-dependent kinase binding activity. Such methods would comprise, for example:

(a) obtaining a cyclin-dependent kinase protein;

(b) contacting the cyclin-dependent kinase protein with a candidate substance; and

(c) determining the binding of the candidate substance to the cyclin-dependent kinase protein.

In yet another embodiment, the assay looks at putative inhibitors of virus replication. Such methods would comprise, for example:

(a) contacting a cell with a virus;

(b) contacting a cell with an inhibitor of cyclin-dependent kinase;

(c) measuring a cellular response; and

(d) measuring the yield of infectious virus.

One skilled in the art may realize that measuring the yield of infectious virus may also include measuring the yield of a virus specific component involved in virus replication.

Assays may be conducted in cell free systems, in isolated cells, or in organisms including transgenic animals.

(ii) Inhibitors and Activators

An inhibitor according to the present invention may be one which exerts an inhibitory effect on the expression or function of cyclin-dependent kinase. By the same token, an activator according to the present invention may be one which exerts a stimulatory effect on the expression or function of cyclin-dependent kinase.

(iii) Candidate Substances

As used herein, the term “candidate substance” refers to any molecule that may potentially modulate cyclin-dependent kinase expression or function. The candidate substance may be a small molecule inhibitor, a protein or fragment thereof, or even a nucleic acid molecule or portions thereof, e.g. nucleoside analogs.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

On the other hand, it may prove to be the case that the most useful pharmacological compounds will be compounds that are structurally related to compounds which interact naturally with cyclin-dependent kinases. Creating and examining the action of such molecules is known as “rational drug design,” and include making predictions relating to the structure of target molecules. Thus, it is understood that the candidate substance identified by the present invention may be a small molecule inhibitor or any other compound (e.g., polypeptide or polynucleotide) that may be designed through rational drug design starting from known inhibitors of cyclin-dependent kinase activity.

The goal of rational drug design is to produce structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule like cyclin-dependent kinase, and then design a molecule for its ability to interact with cyclin-dependent kinase. Alternatively, one could design a partially functional fragment of cyclin-dependent kinase (binding, but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Other suitable inhibitors include antisense molecules, ribozymes, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

B. In vitro Assays

A quick, inexpensive and easy assay to run is a binding assay. Binding of a molecule to a target (e.g., cyclin-dependent kinase) may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a cyclin-dependent kinase molecule or fragment thereof is provided.

A target cyclin-dependent kinase protein may be either free in solution, fixed to a support, expressed in or on the surface of a cell. Either the target CDK protein or the compound may be labeled, thereby permitting determining of binding. In another embodiment, the assay may measure the inhibition of binding of a target CDK protein to a natural or artificial substrate or binding partner. Competitive binding assays can be performed in which one of the agents is labeled. Usually, the target CDK protein will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, cyclin-dependent kinase and washed. Bound polypeptide is detected by various methods.

Purified target, such as cyclin-dependent kinase, can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region (e.g., the C-terminus of cyclin-dependent kinase) to a solid phase.

C. In cyto Assays

Various cell lines that express cyclin-dependent kinase can be utilized for screening of candidate substances. For example, cells containing cyclin-dependent kinase with an engineered indicator can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (e.g., growth, size, or Ca²⁺ effects). Alternatively, molecular analysis may be performed in which the function of cyclin-dependent kinase and related pathways may be explored. This involves assays such as those for protein production, enzyme function, substrate utilization, mRNA expression (including differential display of whole cell or polyA RNA) and others.

D. In vivo Assays

The present invention particularly contemplates the use of various animal models. Transgenic animals may be created with constructs that permit cyclin-dependent kinase expression and activity to be controlled and monitored. The generation of these animals has been described elsewhere in this document.

Treatment of these animals with test compounds (e.g., CDK inhibitors) will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

E. Product ion of Inhibitors

In an extension of any of the previously described screening assays, the present invention also provide for methods of producing inhibitors. The methods comprising any of the preceding screening steps followed by an additional step of “producing the candidate substance identified as a modulator of” the screened activity.

IV. Nucleic Acids

A. Nucleoside Analogs

Nucleoside analogs according to the present invention may be derived from and structurally similar to the nucleosides that are used as the “building blocks” of DNA, but different enough to inhibit DNA synthesis. A nucleoside contains a nucleic acid base and a sugar, e.g., ribose or deoxyribose. Nucleic acid bases are classified as purines or pyrimidines. The purines include adenine or guanine. The prymidines include thymine, cytosine and uracil. According to the present invention, nucleoside analogs may be derived directly or indirectly from natural sources.

B. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

C. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et. al., 1987; Forster and Symons, 1987). For example, a large number of ribozymes accelerate phosphoester transfer reactions with a high degree of specificity, often cleaving only one of several phosphoesters in an oligonucleotide substrate (Cook et. al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et. al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et. al., 1991; Sarver et. al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

D. Nucleic Acids Encoding Cyclin-Dependent Kinase or a Candidate Inhibitor

Nucleic acids according to the present invention may encode an entire cyclin-dependent kinase gene, a domain of cyclin-dependent kinase, or any other fragment of cyclin-dependent kinase as set forth herein. Further, nucleic acids in the present invention may encode an entire candidate CDK inhibitor or any fragment thereof. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given cyclin-dependent kinase or a candidate CDK inhibitor from a given species may be represented by natural variants that have slightly different nucleic acid sequences but, nonetheless, encode the same protein (see Table 1 below).

The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUG AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The DNA segments of the present invention include those encoding biologically functional equivalent cyclin-dependent kinase proteins and peptides or candidate CDK inhibitor proteins or peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

E. Oligonucleotide Probes and Primers

Naturally, the present invention also encompasses DNA segments that are complementary, or essentially complementary, to the nucleic acid sequence of either the cyclin-dependent kinase or a candidate CDK inhibitor protein. As used herein, the term “complementary sequences” means nucleic acid sequences that are substantially complementary, as may be assessed by the same nucleotide comparison set forth above, or as defined as being capable of hybridizing to a nucleic acid segment of cyclin-dependent kinase or a candidate CDK inhibitor under relatively stringent conditions such as those described herein.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3431 bases and longer are contemplated as well. Such oligonucleotides will find use, for example, as probes in Southern and Northern blots and as primers in amplification reactions.

Suitable hybridization conditions will be well known to those of skill in the art. In certain applications, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mm KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to cyclin-dependent kinase or candidate CDK inhibitors or, more particularly, homologs of cyclin-dependent kinase or candidate CDK inhibitors from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides the ability to prepare and test sequence variants, incorporating one or more of the foregoing considerations, by introducing one or more nucleotide sequence changes into the DNA.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

F. Vectors for Cloning, Gene Transfer and Expression

Within certain embodiments, expression vectors are employed to express a cyclin-dependent kinase polypeptide product, which can then be purified and, for example, be used to vaccinate animals to generate antisera or monoclonal antibody with which further studies may be conducted. Further, gene therapy may be used with the candidate CDK inhibitor peptides. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” or “expression cassette” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest (e.g., cyclin-dependent kinase or CDK inhibitor).

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et. al., 1983; Gilles et. al., 1983; Grosschedl et. |
| | al., 1985; Atchinson et. al., 1986, 1987; Imler et. al., |
| | 1987; Weinberger et. al., 1984; Kiledjian et. al., 1988; |
| | Porton et al.; 1990 |
| Immunoglobulin Light Chain | Queen et. al., 1983; Picard et. al., 1984 |
| T-Cell Receptor | Luria et. al., 1987; Winoto et. al., 1989; Redondo et al.; |
| | 1990 |
| HLA DQ a and/or DQ β | Sullivan et. al., 1987 |
| β-Interferon | Goodbourn et. al., 1986; Fujita et. al., 1987; Goodbourn |
| | et. al., 1988 |
| Interleukin-2 | Greene et. al., 1989 |
| Interleukin-2 Receptor | Greene et. al., 1989; Lin et. al., 1990 |
| MHC Class II 5 | Koch et. al., 1989 |
| MHC Class II HLA-DRa | Sherman et. al., 1989 |
| β-Actin | Kawamoto et. Al., 1988; Ng et al.; 1989 |
| Muscle Creatine Kinase (MCK) | Jaynes et. al., 1988; Horlick et. al., 1989; Johnson et. al., |
| | 1989 |
| Prealbumin (Transthyretin) | Costa et. al., 1988 |
| Elastase I | Ornitz et. al., 1987 |
| Metallothionein (MTII) | Karin et. al., 1987; Culotta et. al., 1989 |
| Collagenase | Pinkert et. al., 1987; Angel et. al., 1987 |
| Albumin | Pinkert et. al., 1987; Tronche et. al., 1989, 1990 |
| α-Fetoprotein | Godbout et. al., 1988; Campere et. al., 1989 |
| t-Globin | Bodine et. al., 1987; Perez-Stable et. al., 1990 |
| β-Globin | Trudel et. al., 1987 |
| c-fos | Cohen et. al., 1987 |
| c-HA-ras | Triesman, 1986; Deschamps et. al., 1985 |
| Insulin | Edlund et. al., 1985 |
| Neural Cell Adhesion Molecule | Hirsh et. al., 1990 |
| (NCAM) | |
| α₁-Antitrypain | Latimer et. al., 1990 |
| H2B (TH2B) Histone | Hwang et. al., 1990 |
| Mouse and/or Type I Collagen | Ripe et. al., 1989 |
| Glucose-Regulated Proteins | Chang et. al., 1989 |
| (GRP94 and GRP78) | |
| Rat Growth Hormone | Larsen et. al., 1986 |
| Human Serum Amyloid A (SAA) | Edbrooke et. al., 1989 |
| Troponin I (TN I) | Yutzey et. al., 1989 |
| Platelet-Derived Growth Factor | Pech et. al., 1989 |
| (PDGF) | |
| Duchenne Muscular Dystrophy | Klamut et. al., 1990 |
| SV40 | Banerji et. al., 1981; Moreau et. al., 1981; Sleigh et. al., |
| | 1985; Firak et. al., 1986; Herr et. al., 1986; Imbra et. al., |
| | 1986; Kadesch et. al., 1986; Wang et. al., 1986; Ondek et. |
| | al., 1987; Kuhl et. al., 1987; Schaffner et. al., 1988 |
| Polyoma | Swartzendruber et. al., 1975; Vasseur et. al., 1980; |
| | Katinka et. al., 1980, 1981; Tyndell et. al., 1981; Dandolo |
| | et. al., 1983; de Villiers et. al., 1984; Hen et. al., 1986; |
| | Satake et. al., 1988; Campbell and/or Villarreal, 1988 |
| Retroviruses | Kriegler et. Al., 1982, 1983; Levinson et. al., 1982; |
| | Kriegler et. al., 1983, 1984a, b, 1988; Bosze et. al., 1986; |
| | Miksicek et. al., 1986; Celander et. al., 1987; Thiesen et. |
| | al., 1988; Celander et. al., 1988; Choi et. al., 1988; |
| | Reisman et. al., 1989 |
| Papilloma Virus | Campo et. al., 1983; Lusky et. al., 1983; Spandidos and/or |
| | Wilkie, 1983; Spalholz et. al., 1985; Lusky et. al., 1986; |
| | Cripe et. al., 1987; Gloss et. al., 1987; Hirochika et. al., |
| | 1987; Stephens et. al., 1987; Glue et. al., 1988 |
| Hepatitis B Virus | Bulla et. al., 1986; Jameel et. al., 1986; Shaul et. al., 1987; |
| | Spandau et. al., 1988; Vannice et. al., 1988 |
| Human Immunodeficiency Virus | Muesing et. al., 1987; Hauber et. al., 1988; Jakobovits et. |
| | al., 1988; Feng et. al., 1988; Takebe et. al., 1988; Rosen |
| | et. al., 1988; Berkhout et. al., 1989; Laspia et. al., 1989; |
| | Sharp et. al., 1989; Braddock et. al., 1989 |
| Cytomegalovirus (CMV) | Weber et. al., 1984; Boshart et. al., 1985; Foecking et. al., |
| | 1986 |
| Gibbon Ape Leukemia Virus | Holbrook et. al., 1987; Quinn et. al., 1989 |

TABLE 3
Inducible Elements

| Element | Inducer | References |
|---|---|---|
| MT II | Phorbol Ester (TFA) | Palmiter et. al., 1982; Haslinger |
| | Heavy metals | et. al., 1985; Searle et. al., 1985; |
| | | Stuart et. al., 1985; Imagawa et. |
| | | al., 1987, Karin et. al., 1987; |
| | | Angel et. al., 1987b; McNeall et. |
| | | al., 1989 |
| MMTV (mouse mammary | Glucocorticoids | Huang et. al., 1981; Lee et. al., |
| tumor virus) | | 1981; Majors et. al., 1983; |
| | | Chandler et. al., 1983; Lee et. al., |
| | | 1984; Ponta et. al., 1985; Sakai et. |
| | | al., 1988 |
| β-Interferon | poly(rI)x | Tavernier et. al., 1983 |
| | poly(rc) | |
| Adenovirus 5 E2 | ElA | Imperiale et. al., 1984 |
| Collagenase | Phorbol Ester (TPA) | Angel et. al., 1987a |
| Stromelysin | Phorbol Ester (TPA) | Angel et. al., 1987b |
| SV40 | Phorbol Ester (TPA) | Angel et. al., 1987b |
| Murine MX Gene | Interferon, Newcastle | Hug et. al., 1988 |
| | Disease Virus | |
| GRP78 Gene | A23187 | Resendez et. al., 1988 |
| α-2-Macroglobulin | IL-6 | Kunz et. al., 1989 |
| Vimentin | Serum | Rittling et. al., 1989 |
| MHC Class I Gene H-2 κb | Interferon | Blanar et. al., 1989 |
| HSP70 | ElA, SV40 Large T Antigen | Taylor et. al., 1989, 1990a, 1990b |
| Proliferin | Phorbol Ester-TPA | Mordacq et. al., 1989 |
| Tumor Necrosis Factor | PMA | Hensel et. al., 1989 |
| Thyroid Stimulating | Thyroid Hormone | Chatterjee et. al., 1989 |
| Hormone α Gene | | |

(ii) Selectable Markers

In certain embodiments of the invention, the cells contain nucleic acid constructs of the present invention. A cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Multigene Constructs and IRES

In certain embodiments of the invention, the use of internal ribosome binding sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

(iv) Polyadenylation Signals

In expression, one will typically include a polyadenylation signal to effect proper polyadenylation of the transcript. In specific embodiments where a cDNA insert is employed, it may be desirable to include a polyadenylation site. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

(v) Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et. al., 1988 and Ausubel et. al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

(vi) Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

(vii) Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

(viii) Delivery of Expression Vectors

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Several non-viral methods for the transfer of expression constructs into cultured mammalian cells are contemplated by the present invention. These include calcium phosphate precipitation (Graham and Van Der Eb, 1973; Chen and Okayama, 1987; Rippe et. al., 1990) DEAE-dextran (Gopal, 1985), electroporation (Tur-Kaspa et. al., 1986; Potter et. al., 1984), direct microinjection (Harland and Weintraub, 1985), DNA-loaded liposomes (Nicolau and Sene, 1982; Fraley et. al., 1979) and lipofectamine-DNA complexes, cell sonication (Fechheimer et. al., 1987), gene bombardment using high velocity microprojectiles (Yang et. al., 1990), and receptor-mediated transfection (Wu and Wu, 1987; Wu and Wu, 1988). Some of these techniques may be successfully adapted for in vivo or ex vivo use.

The expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et. al., 1987). Several devices for accelerating small particles have been developed. One such device relies on a high voltage discharge to generate an electrical current, which in turn provides the motive force (Yang et. al., 1990). The microprojectiles used have consisted of biologically inert substances such as tungsten or gold beads.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et. al., 1989). In other embodiments, the liposome may be complexed or employed in conjunction with nuclear non-histone chromosomal proteins (HMG-1) (Kato et. al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et. al., 1990). Recently, a synthetic neoglycoprotein, which recognizes the same receptor as ASOR, has been used as a gene delivery vehicle (Ferkol et al., 1993; Perales et. al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et. al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

Another method for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus vectors have been used in eukaryotic gene expression (Levrero et. al., 1991; Gomez-Foix et. al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et. al., 1990; Rich et. al., 1993). Studies in administering recombinant adenovirus to different tissues include trachea instillation (Rosenfeld et. al., 1991; Rosenfeld et. al., 1992), muscle injection (Ragot et. al., 1993), peripheral intravenous injections (Herz and Gerard, 1993) and stereotactic inoculation into the brain (Le Gal La Salle et. al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

A different approach to targeting of recombinant retroviruses was designed in which biotinylated antibodies against a retroviral envelope protein and against a specific cell receptor were used. The antibodies were coupled via the biotin components by using streptavidin (Roux et. al., 1989). Using antibodies against major histocompatibility complex class I and class II antigens, they demonstrated the infection of a variety of human cells that bore those surface antigens with an ecotropic virus in vitro (Roux et. al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et. al., 1981). Another concern with the use of defective retrovirus vectors is the potential appearance of wild-type replication-competent virus in the packaging cells. This can result from recombination events in which the intact- sequence from the recombinant virus inserts upstream from the gag, pol, env sequence integrated in the host cell genome. However, new packaging cell lines are now available that should greatly decrease the likelihood of recombination (Markowitz et. al., 1988; Hersdorffer et. al., 1990).

Other viral vectors may be employed as expression constructs in the present invention. Vectors derived from viruses such as vaccinia virus (Ridgeway, 1988; Baichwal and Sugden, 1986; Coupar et. al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et. al., 1988; Horwich et. al., 1990).

V. Proteins and Peptides

The present invention relates to the entire protein or fragments of the polypeptide (cyclin-dependent kinase or CDK inhibitor) that may or may not retain the various functions described below. Fragments, including the N-terminus of the molecule may be generated by genetic engineering of translation stop sites within the coding region (discussed below). Alternatively, treatment of the polypeptides with proteolytic enzymes, known as proteases, can produce a variety of N-terminal, C-terminal and internal fragments. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

Another embodiment for the preparation of polypeptides according to the invention is the use of peptide mimetics. Mimetics are peptide-containing molecules that mimic elements of protein secondary structure (Johnson et al., 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of cyclin-dependent kinase, but with altered and even improved characteristics. Also contemplated is the development of a peptide mimetic of a CDK inhibitor with improved activity to inhibit cyclin-dependent kinase.

A. Variants of Protein

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity (e.g. CDK or CDK inhibitor activity), as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9) and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

As outlined above, amino acid substitutions are generally based on the relative similarity of the amino acid side-chain substituents, for example, their hydrophobicity, hydrophilicity, charge, size, and the like. Exemplary substitutions that take the foregoing characteristics into consideration are well known to those of skill in the art and include: arginine and lysine; glutamate and aspartate; serine and threonine; glutamine and asparagine; and valine, leucine and isoleucine.

B. Domain Switching

Domain switching involves the generation of chimeric molecules using different but, in this case, related polypeptides. By comparing various cyclin-dependent kinase proteins, one can make predictions as to the functionally significant regions of these molecules. It is possible, then, to switch related domains of these molecules in an effort to determine the criticality of these regions to cyclin-dependent kinase function. These molecules may have additional value in that these “chimeras” can be distinguished from natural molecules, while possibly providing the same function. Further, these “chimeras” may be used as CDK inhibitors providing that there is a loss of cyclin-dependent kinase activity.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. Fusion proteins may be useful in the development of gene therapy to specifically target the candidate CDK inhibitor proteins.

D. Purification of Proteins

It may be desirable to purify cyclin-dependent kinase or candidate CDK inhibitor proteins or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et. al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Synthetic Peptides

The present invention also includes smaller candidate CDK inhibitor-related peptides for use in various embodiments of the present invention. The peptides of the invention can also be synthesized in solution or on a solid support in accordance with conventional techniques. Various automatic synthesizers are commercially available and can be used in accordance with known protocols. See, for example, Stewart and Young, (1984); Tam et. al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

VI. Generating Antibodies Reactive with Cyclin-Dependent Kinase

In another aspect, the present invention contemplates an antibody that is immunoreactive with a cyclin-dependent kinase molecule of the present invention, or any portion thereof. An antibody can be a polyclonal or a monoclonal antibody. In a preferred embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a cyclin-dependent kinase polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistocliemical procedures such as tissue staining, as well as in other procedures which may utilize antibodies specific to cyclin-dependent kinase-related antigen epitopes. Additionally, it is proposed that monoclonal antibodies specific to the particular cyclin-dependent kinase of different species may be utilized in other useful applications

In general, both polyclonal and monoclonal antibodies against cyclin-dependent kinase may be used in a variety of embodiments. For example, they may be employed in antibody cloning protocols to obtain cDNAs or genes encoding other cyclin-dependent kinases. They may also be used in inhibition studies to analyze the effects of cyclin-dependent kinases related peptides in cells or animals. Cyclin-dependent kinase antibodies will also be useful in immunolocalization studies to analyze the distribution of cyclin-dependent kinases during various cellular events, for example, to determine the cellular or tissue-specific distribution of cyclin-dependent kinases polypeptides under different points in the cell cycle. A particularly useful application of such antibodies is in purifying native or recombinant cyclin-dependent kinases, for example, using an antibody affinity column. The operation of all such immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are given in the examples below.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified cyclin-dependent kinase protein, polypeptide or peptide or cell expressing high levels of cyclin-dependent kinase. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

VII. Immunologic Analysis

The use of antibodies of the present invention, in an ELISA assay is contemplated. For example, anti-cyclin-dependent kinase antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for cyclin-dependent kinase or a fragment thereof that differs from the first antibody. Appropriate conditions preferably include diluting the sample with dilutents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

VIII. Mutagenesis

Where employed, mutagenesis will be accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

A. Random Mutagenesis

(i) Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations (Oppenheimer et al. 1991). Insertion mutagenesis has been very successful in bacteria and Drosophila (Cooley et al. 1988) and recently has become a powerful tool in corn (Schmidt et al. 1987); Arabidopsis; (Marks et. al., 1991; Koncz et al. 1990); and Antirrhinum (Sommer et al. 1990).

Transposable genetic elements are DNA sequences that can move (transpose) from one place to another in the genome of a cell. The first transposable elements to be recognized were the Activator/Dissociation elements of Zea mays (McClintock, 1957). Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini. These terminal inverted repeats may be anything from a few bases to a few hundred bases long and in many cases they are known to be necessary for transposition.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another. Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are refelTed to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

(ii) Chemical Mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test (McCann et. al., 1975) which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

(iii) Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman et. al., 1989). Synthesis of cyclin and co-regulated polypeptides is suppressed by ionizing radiation in rat REF52 cells, but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte, et. al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

(iv) In vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et. al., 1989).

In recent years, techniques for estimating the equilibrium constant for ligand binding using minuscule amounts of protein have been developed (Blackburn et. al., 1991; U.S. Pat. Nos. 5,221,605 and 5,238,808). The ability to perform functional assays with small amounts of material can be exploited to develop highly efficient, in vitro methodologies for the saturation mutagenesis of antibodies. Cloning steps may be bypassed by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et. al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

(v) Random Mutagenesis by Fragmentation and Reassembly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

B. Site-Directed Mutagenesis

Structure-guided site-specific mutagenesis represents a powerful tool for the dissection and engineering of protein-ligand interactions (Wells, 1996, Braisted et. al, 1996). The technique provides for the preparation and testing of sequence variants by introducing one or more nucleotide sequence changes into a selected DNA.

Site-specific mutagenesis uses specific oligonucleotide sequences, which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et. al., 1996, Brown et. al., 1996; Zeng et. al., 1996; Burton and Barbas, 1994; Yelton et. al., 1995; Jackson et. al., 1995; Short et. al., 1995; Wong et. al., 1996; Hilton et. al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

IX. Methods of Making Transgenic Mice

A particular embodiment of the present invention provides transgenic animals that contain cyclin-dependent kinase-related constructs. Transgenic animals expressing cyclin-dependent kinase recombinant cell lines derived from such animals, and transgenic embryos may be useful in determining the exact role that candidate CDK inhibitors play on suppression of viral infections by affecting the activity of cyclin-dependent kinase. The use of constitutively expressed cyclin-dependent kinase provides a model for over- or unregulated expression.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris,pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); in The Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate with a 5 IU injection (0.1 cc, ip) of pregnant mare serum gonadotropin (PMSG; Sigma) followed 48 hrs later by a 5 IU injection (0.1 cc, ip) of human chorionic gonadotropin (hCG; Sigma). Females are placed with males immediately after hCG injection. Twenty-one hrs after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

X. Drug Formulations and Administration

Where clinical applications are contemplated, it will be necessary to prepare pharmaceutical compositions (e.g., expression vectors, recombinant cells, candidate CDK inhibitors or analogs thereof) in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. The delivery of CDK inhibitors as DNA plasmid may be linked to polycations, which are water-soluble complexes and known and used in the art as a delivery system for DNA plasmids. This strategy employs the use of a soluble system, which will convey the DNA into the cells via a receptor-mediated endoytosis (Wu & Wu 1988). Specific ligands or receptors must be conjugated to a polycation. Buffers also will be employed when recombinant cells are introduced into an animal.

Aqueous compositions of the present invention comprise an effective amount of the vector, cells or CDK inhibitor or analog thereof, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances are well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The pharmaceutical forms of CDK inhibitors or analogs suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, and the like. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Prolonged absorption of the injectable compositions can be brought about by the use in the compositions of agents delaying absorption, for example, aluminum monostearate and gelatin.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

In a preferred embodiment for parenteral administration, the solution should be suitably buffered as necessary for the stability of the CDK or analog active ingredient and the liquid diluent first rendered isotonic with sufficient saline or glucose. Preferred pH range of the solution will be between 6.5 and 7.5. These particular aqueous solutions are especially suitable for intraperitoneal administration. In this connection, sterile aqueous media which can be employed will be known to those of skill in the art in light of the present disclosure.

The preferred dosage of CDK inhibitor in a parenteral administration may vary, depending upon the extent of the virus infection, the severity of the symptoms associated with the infection and patient age, weight and medical history. The number of administrations of the parenteral composition of the CDK inhibitor will also vary according to the response of the individual patient to the treatment. In one exemplary application, the dosage of CDK inhibitor may vary with the type of disease and the route of administration. Further studies with animal models of infection will completely define projected doses. A dose of 3 μg/kg is tolerated by rate and it is expected that for humans, a roscovitine dose of 1 μg/kg to 10 μg/kg will be tolerated and antivirally effective. For example, the dose, to be prophylactically or therapeutically effective should be enough to achieve a 4 μM to 20 μM roscovitine concentration in the environment of infected or potentially infected cells. Weaker CDK2 inhibitors will be needed at higher concentrations, and stronger, at lower.

In other preferred embodiments of the invention, pharmacologically active compositions could be introduced to the patient through transdermal delivery of a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture.

Suitable amounts of the active ingredient of the CDK inhibitor that may be used in the compositions for topical administration may range from 0.1-100 μg per 1000 g of the composition. Administration of the ointments, creams and lotions of this invention may be from between once a day to as often as is necessary to relieve symptoms and will vary according to the strength of the medication, active ingredient, patient age and the severity of the symptoms. Administration of the topical medications of this invention may be directly to the infected area.

Another preferred method of administering pharmacologically active compositions of CDK inhibitors is as an aerosol. Aerosol compositions of the CDK inhibitor may be especially useful for the treatment of living tissue, although they could also be used for dermal applications. The term aerosol refers to a colloidal system of finely divided solid of liquid particles dispersed in a liquified or pressurized gas propellant. The typical aerosol of the present invention for oral or nasal inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to patient age, weight and the severity and response of the symptoms.

For oral administration the CDK inhibitors of the present invention may be incorporated with excipients and used in the form of non-ingestible mouthwashes and dentifrices. A mouthwash may be prepared incorporating the CDK inhibitors in the required amount in an appropriate solvent, such as a sodium borate solution (Dobell's Solution). Alternatively, the CDK inhibitors may be incorporated into an antiseptic wash containing sodium borate, glycerin and potassium bicarbonate. The CDK inhibitors may also be dispersed in dentifrices, including: gels, pastes, powders and slurries. The CDK inhibitors may be added in a therapeutically effective amount to a paste dentifrice that may include water, binders, abrasives, flavoring agents, foaming agents, and humectants.

XI. Lipid Formulations and/or Nanocapsules

In certain embodiments, the use of lipid formulations and/or nanocapsules is contemplated for the introduction of CDK inhibitor compounds or pharmaceutically acceptable salts thereof or CDK inhibitor protein, polypeptides, peptides and/or agents, and/or gene therapy vectors, including both wild-type and/or antisense vectors, into host cells.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and/or such particles may be easily made.

In a preferred embodiment of the invention, the CDK inhibitor may be associated with a lipid. The CDK inhibitor associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/CDK inhibitor associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes.

Phospholipids may be used for preparing the liposomes according to the present invention and may carry a net positive, negative, or neutral charge. Diacetyl phosphate can be employed to confer a negative charge on the liposomes, and stearylamine can be used to confer a positive charge on the liposomes. The liposomes can be made of one or more phospholipids.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Phospholipids can form a variety of structures other than liposomes when dispersed in water, depending on the molar ratio of lipid to water. At low ratios the liposome is the preferred structure. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs.

Liposomes interact with cells via four different mechanisms: endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Liposome-mediated oligonucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hrs, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

A pharmaceutical composition comprising the liposomes will usually include a sterile, pharmaceutically acceptable carrier or diluent, such as water or saline solution.

XII. Gene Therapy Administration

One skilled in the art may recognize that the mode of DNA delivery of this invention could potentially be used to deliver DNA to specific cells for gene therapy. For gene therapy, a skilled artisan will be cognizant that the vector to be utilized must contain the gene of interest operatively limited to a promoter. For antisense gene therapy, the antisense sequence of the gene of interest would be operatively linked to a promoter. One skilled in the art recognizes that in certain instances other sequences such as a 3′ UTR regulatory sequences are useful in expressing the gene of interest. Where appropriate, the gene therapy vectors can be formulated into preparations in solid, semisolid, liquid or gaseous forms in the ways known in the art for their respective route of administration. Means known in the art can be utilized to prevent release and absorption of the composition until it reaches the target organ or to ensure timed release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds. A sufficient amount of vector containing the therapeutice nucleic acid sequence must be administered to provide a pharmacologically effective dose of the gene product.

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein said vector is complexed to another entity, such as a liposome, aggregated protein or transporter molecule.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody mediated detection, mRNA or protein half life studies, or particularized assays to detect protein or polypeptide encoded by the transferred nucleic acid, or impacted in level or function due to such transfer).

These methods described herein are by no means all inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

It is possible that cells containing the therapeutic gene may also contain a suicide gene (i. e., a gene, which encodes a product that can be used to destroy the cell, such as herpes simplex virus thymidine kinase). In many gene therapy situations, it is desirable to be able to express a gene for therapeutic purposes in a host cell but also to have the capacity to destroy the host cell once the therapy is completed, becomes uncontrollable, or does not lead to a predictable or desirable result. Thus, expression of the therapeutic gene in a host cell can be driven by a promoter although the product of said suicide gene remains harmless in the absence of a prodrug. Once the therapy is complete or no longer desired or needed, administration of a prodrug causes the suicide gene product to become lethal to the cell. Examples of suicide gene/prodrug combinations which may be used are Herpes Simplex Virus-thymidine kinase (HSV-tk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

Those of skill in the art are well aware of how to apply gene delivery to in vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below. Various routes are contemplated, but local provision to the heart and systemic provision (intraarterial or intravenous) are preferred.

XIII. Combined Therapy

In another embodiment, it is envisioned to use a candidate CDK inhibitor in combination with other antiviral agents. These antiviral agents may include traditional antiviral agents, however, it may also include nontraditional compounds, e.g., antineoplastics. Examples of traditional antiviral agents include, but are not limited to, aciclovir, ganciclovir, famciclovir, cidofovir, vidarabine, idoxuridine, foscarnet, triflyorothymidine, vidarabine, DHPG (9-(1,3-dihydroxy-2-propoxymethyl)guanine), AZT (3′-axido-3′ deoxythymidine), lamivudine or phosphonoacetic acid. Further, it is envisioned that an antiviral agent that has poor activity, but minimal toxicity, may be combined with a CDK inhibitor to block virus replication. It is conceiveable that an antiviral agent that has poor activity inhibits at least one pathway of virus replication, however, the virus is able to utilize another pathway, resulting in the appearance of poor antiviral activity of the agent. If a CDK inhibitor marginally blocks another pathway in virus replication, then it is contemplated that the combination of the poor antiviral agent and the CDK inhibitor would efficiently block virus replication by blocking multiple pathways of the virus.

Combinations may be achieved by contacting cells with a single composition or pharmacological formulation that includes both agents (CDK inhibitor compounds and antiviral compounds), or by contacting the cell with two distinct compositions or formulations, at the same time. Alternatively, administeration of one agent may precede or follow the treatment with a second agent by intervals ranging from minutes to weeks. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a CDK inhibitor or the other agent will be desired. Various combinations may be employed, where CDK inhibitor is “A” and the other agent or antiviral compound is “B”, as exemplified below:

A/B/A B/A/B B/B/A A/A/B B/A/A A/B/B B/B/B/A B/B/A/B

A/A/B/B A/B/A/B A/B/B/A B/B/A/A B/A/B/A B/A/A/B B/B/B/A

A/A/A/B B/A/A/A A/B/A/A A/A/B/A A/B/B/B B/A/B/B B/B/A/B

Other combinations are contemplated as well.

### XIV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cell Culture

Mammalian cells, e.g., human diploid embryonic lung fibroblasts (LU) (Albrecht et. al., 1980a), passage 12-20, or U-373 MG astrocytoma cells, were cultured in Eagle's Minimum Essential Medium with Earle's salts (EMEM) with fetal bovine serum (FBS) and penicillin (100 units/ml)/streptomycin (100 μ/ml) at 37° C. in a 5% CO₂ atmosphere. To obtain serum-arrested cultures, cells were grown to 70-80% confluence. The medium was then removed and the cells were washed with warm, serum-free EMEM. After washing, fresh, serum-free EMEM was placed on the cells and the incubation continued for another 48 hr to achieve growth factor arrest. After serum-deprivation, the serum-free medium was decanted and reserved. The cells were either infected with a virus, mock-infected, or stimulated with FBS in fresh EMEM. To obtain density-arrested cultures, the cells were initially grown to confluence. The medium was replaced with fresh EMEM containing FBS, and the cells were incubated another 48 hr to insure strict density arrest.

### Example 2

### Virus Propagation

Virus stocks (Human cytomegalovirus strain AD169 (passage 86-92)) were prepared by infecting confluent monolayers of cells at a multiplicity of infection (M.O.I.) of 0.002 plaque forming units (PFU)/cell. Infected cultures were maintained in EMEM containing fetal bovine serum (FBS), and frozen 11-15 days post-infection. Viral stocks were prepared by releasing the virus from the cell by freeze-thaw and/or sonication (2×30 sec). Virus was dispensed into replicative vials and stored at −80° C. Before use the cellular debris was removed by sedimentation.

### Example 3

### Virus Infectivity Assay

Virus infectivity was determined as described previously by Albrecht and Weller (1980). Briefly, confluent cell monolayers in 35 mm dishes were infected with 10-fold serial dilutions of virus stock at 37° C. for 1 hr. The virus inoculum was removed and replaced with 1.5 ml of agarose overlay containing EMEM, FBS, agarose, and sodium bicarbonate. After 7 days incubation an additional 1.5 ml of overlay was added and the dishes incubated 7 more days. After 14 days total incubation, cells were fixed with 10% formalin and stained with 0.03% methylene blue and plaques counted with the aid of a dissecting microscope.

### Example 4

### Virus Infection

Virus stock was added to a calculated multiplicity of infection of 5 PFU/cell. The virus inoculum or mock-infecting fluids were removed after 1 hr. For infection of subconfluent cultures, the cells were maintained after removal of the virus in the reserved “spent” serum-free medium. For confluent cells, the virus inoculum and mock-infecting fluids were removed and replaced with warm EMEM containing FBS. For mock infection, cells were exposed to mock-infecting fluids (Boldogh et al., 1990) containing no virus particles for 1 hr.

### Example 5

### Treatment of Infected Cells

Confluent monolayers of cells were infected at a M.O.I. of 5 PFU/cell and allowed to absorb for 1 hr at 37° C. The virus inoculum was removed and replaced with warm EMEM containing FBS and the indicated concentration of drug or vehicle. Cells were harvested 96 hr post-infection and assayed for infectivity as described above in Example 3, or cells were harvested 72 hr post-infection and HCMV DNA abundance determined by slot blot hybridization.

### Example 6

### Virus Purification

Virus particles were pelleted by sedimentation from clarified virus stocks by centrifugation at 100,000×g for 90 minutes at room temperature. Following centrifugation the supernatant was decanted and reserved (virus-free supernatant). The pelleted virus was resuspended in serum free EMEM and subsequently used for infection.

### Example 7

### UV Irradition of Virus

To inhibit viral gene expression, virus stocks were UV-irradiated on an ice bed at 254 nm at a dose rate of 8×10⁻⁶ J/s/mm², for 30 min as described previously (Boldogh et al., 1990). Under these conditions viral gene expression was abolished (Boldogh et. al., 1990). To ensure that UV-irradiation protocol inhibited viral gene expression, cells were infected with HCMV or UV-irradiated HCMV and stained for the expression of HCMV immediate early (IE) other proteins.

### Example 8

### Flow Cytometry

Cells were harvested by trypsinization at selected times after virus infection, mock infection, or serum stimulation. The cells were washed in PBS, collected by sedimentation, suspended in low salt buffer [3% polyethylene glycol, propidium iodine (5 μg/ml), 0.1% Triton-X, 4 mM sodium citrate, RNase A (100 μg/ml, added just before use)], and incubated 20 min at 37° C. High salt buffer [3% polyethylene glycol, propidium iodine (5 μg/ml), 0.1% Triton X-100, 400 mM NaCl] was added, and the cells were maintained at 4° C. overnight. The cellular DNA content was analyzed using a flow cytometer.

### Example 9

### Isotopic Labeling, Isolation and Analysis of DNA

Subconfluent, serum-arrested cells were pulse-labeled for 6 hr with 10 mCi/ml [³H]methyl thymidine (52.74 Ci/mmole). At the end of the pulse, the cultures were rapidly frozen and thawed for 2 cycles to dislodge the cells. DNA was released and analyzed by isopycnic centrifugation as described previously (Albrecht et al., 1980b).

### Example 10

### Western Blotting

Cells were harvested by trypsinization, collected by sedimentation, and lysed in NP-40 lysis buffer. Cellular debris was removed by sedimentation and the supernatant fluids reserved. The protein concentration was determined by the method of Bradford (Bradford, 1976). Equal amounts of protein were resolved by electrophoresis in the presence of SDS on polyacrylamide gels (SDS-PAGE). Proteins were transferred to nitrocellulose membrane (Bio-Rad) and probed with specified antibodies. Immunoreactive proteins were detected by the ECL chemiluminescent system (Amersham), and specific bands were quantified by densitometry.

### Example 11

### Indirect Immunofluorescence

Cells were cultured on sterile glass coverslips. The cells were washed three times in PBS and fixed in acetone:methanol (1:1) at −20° C. for 10 min. The permeabilized cells were incubated with primary antibody diluted in PBS for 1 hr at 37° C. in a humidified chamber. After 2 washes in PBS for 15 min, the cells were incubated with a secondary antibody (affinity-purified, goat anti-mouse or rabbit FITC-conjugated IgG) for 45 min. The excess conjugate was removed by washing the cells in PBS for 30 min. After drying, the cells were mounted in PBS/glycerol (1:1) and examined with the aid of a Zeiss Photomicroscope III using a 40/1.0 Neofluar lens. Images were photographed on slide film.

### Example 12

### Histone H1 Kinase Assay

Kinase assays were accomplished as described previously (Dulic et. al., 1992). Briefly, aliquots containing 150 μg of protein were incubated with antibody for 2 hr at 4° C. The protein/antibodies complexes were then precipitated using Protein A-Sepharose beads. The pellets were washed 3 times with NP-40 lysis buffer, followed by washing 3 times with 2×kinase buffer (40 mM Tris-HCl, pH 7.5, 8 mM MgCl₂). Kinase reactions were undertaken in tubes containing the precipitates in a total volume of 5 μl, which included 3 μl 2× kinase buffer containing 2.5 μg histone H1(GIBCO BRL) and 2 μl (4 μCi), [γ-32P] ATP (10 Ci/mmol, DuPont NEN) at 37° C. for 30 min. The reaction was stopped by the adding 5 μl 2×sample buffer and boiling for 5 min. Each sample was then separated by SDS-PAGE following standard protocols well-known to the skilled artisan. The gels were dried and exposed to film. Specific bands were quantified by densitometry.

### Example 13

### Transient Transfections

Cells were split 24 hr prior to transfection into 100 mm dishes. Cells were transfected with Tfx-50 lipofection reagent (Promega) at a 3:1 lipofectin:DNA ratio for 2 hr. Cells were either removed with trypsin and seeded into 35 mm dishes containing sterile glass coverslips, and cultured 24 hr before being infected as described above, or cells were harvested 48 hr after transfection and processed for western blotting and kinase assays. Two plasmids that were used for transient transfections were pCMVCdk2-wt-HA or pCMVCdk2-dn-HA.

### Example 14

### Slot Blot Hybridization

Total DNA from cells was isolated by phenol extraction as described by Boldogh et al, 1990. Equal aliquots of DNA (2 μg) were heated to 95° C. and transferred to Hybond+ (Amersham) membranes in 10×SSC buffer using a slot blot apparatus. Membranes were denatured for 5 min in 0.5M NaOH-1.5M NaCl buffer and neutralized in 1.5M NaCl, 0.5M Tris-HCl pH. 7.2, 0.001 M EDTA buffer for 5 min, and then dried for 10 min in a vacuum oven at 80° C. Filters were prehybridized in Rapid-Hyb buffer (Amersham) for 3 hr at 60° C. Hybridization was carried out by overnight incubation in the same buffer at 60° C. Probes specific to the DNA were labeled using standard procedures well known in the art. Filters were washed twice in 0.1% SDS in 2×SSC at room temperature for 15 minutes, then 0.1% SDS in 0.2×SSC at 60° C. for 20 min and exposed to film (Kodak XAR-5) at −80° C. One probe that was used was a 253 bp PCRTm amplified immediate early fragment from HCMV strain AD169 that was ³²P labeled by random priming (Promega) as described by the manufacturer.

### Example 15

### Isolation of Nuclei

Cells were washed with PBS and removed from the flask by scraping. Cells were sedimented by centrifugation and resuspended in 1 ml of PBS and transferred to a 1.5 ml centrifuge tube. The cells were again centrifuged and the supernatant removed by suction. The pellet was resuspended in lysis buffer by pipetting and vortexing so no cell aggregates were present. NP-40 was added to a final concentration of 0.5% and the suspension mixed by inversion for 3-5 minutes. Nuclei were sedimented by centrifugation and the cytoplasm (supernatant) fraction reserved. The nuclei pellet was resuspended in 1 ml PBS and again sedimented and the supernatant removed by suction. The pellet was resuspended in lysis buffer and SDS was added to a final concentration of 0.5% and mixed by inversion for 5 min. The nuclear lysates were then clarified by centrifugation (60,000×g at 2° C. for 20 min). The supernatant removed and saved for western blotting.

### Example 16

### Image Processing

Chemilumenescent samples were exposed for intervals that assured linearity of response, as determined by standardization. All radiographic films were analyzed using the Applied Imaging Lynx 5000 digital work station with Lynx V5.5 software. The images were quantified and recorded as tagged image format files (TIFF). The TIFF were used to prepare the graphic images.

### Example 17

### Hematoxylin-eosin Stating

Cells were prepared on glass coverslips inserted into flat bottom 35 mm dishes. At appropriate times following the initiation of virus infection and incubation, infected and control coverslip cultures were removed, rinsed three times in PBS and placed in Bouin's picric acid fixative. The cells remained in the fixative from 1 hr to overnight, at which time the coverslips were transferred to 70% ethanol for a minimum of 24 hr. The fixed cells then were rehydrated in decreasing concentrations of ethanol (5 min each) and placed in Harris' hematoxylin (15 min). The hematoxylin-stained coverslips were destained briefly in 0.4% hydrochloric acid, rinsed in distilled water, and placed in Scott's blueing solution (0.1% lithium carbonate) for 5 min. Following dehydration in increasing concentrations of ethanol (5 min each), the coverslips were placed in alcoholic eosin solution (10 min). After complete dehydration in absolute ethanol (total 20 min) and xylene (total of 20 min), the coverslips were mounted in cytoseal mounting medium and allow to dry.

### Example 18

### Formation of Cyclin E/CDK2 Complexes, Induction of Cyclin E, and Activation of Cyclin E-dependent Kinase

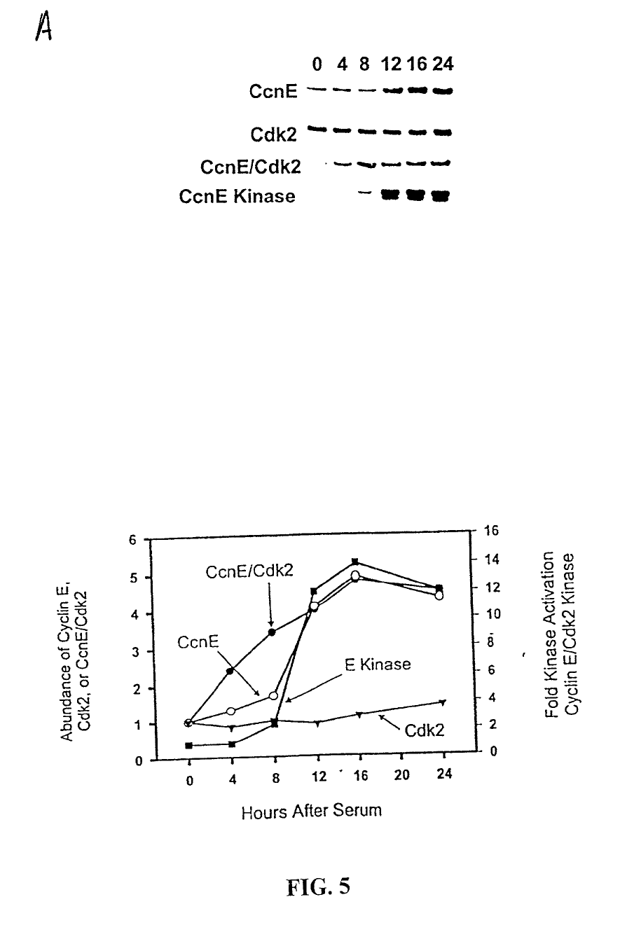

The studies were carried out in serum-arrested human diploid fibrobroblasts stimulated with serum to enter into the cell cycle. These cells exhibited synchronous progression through GI following serum stimulation as illustrated by the data shown in FIG. 5A. The majority of these cells maintain a G1 DNA content for 16 hr after addition of serum. Bromodeoxyuridine (BrDU) labeling of such cultures indicated that <5% of the cells had accumulated detectable amounts of BRdU-labeled DNA within 16 hr after stimulation. The cells rapidly entered S phase between 16 and 24 hr after addition of serum. As shown in FIG. 5A, at least 70% of the cells exhibited >2N DNA content at 24 hr, and BRdU labeling studies indicated that >85% of the cells in these cultures initiated DNA replication between 16 and 24 hr.

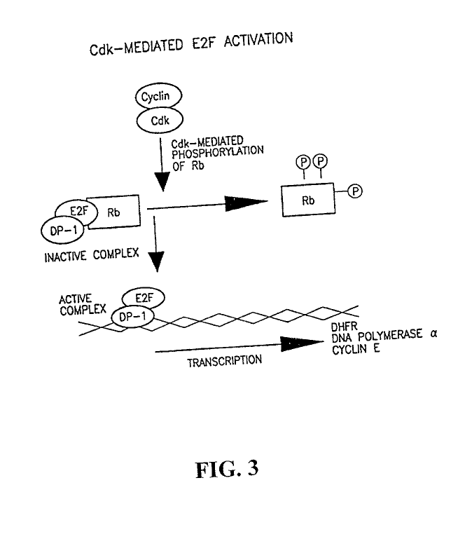

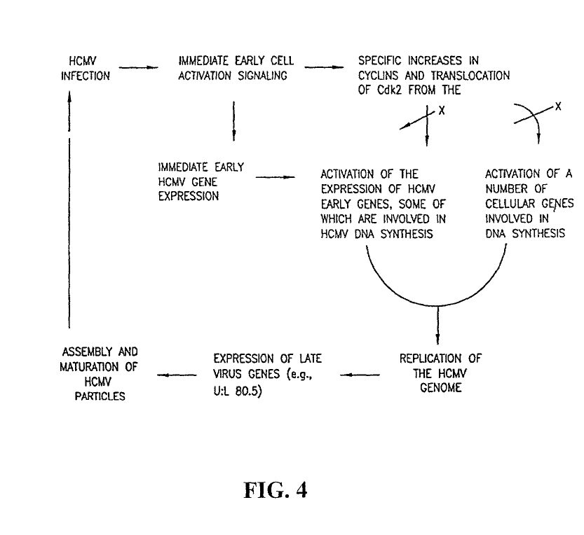

Cyclin E protein was induced after serum stimulation of quiescent human diploid fibroblasts, as shown in FIG. 5A. FIG. 5A contains data from a representative study, and FIG. 3B contains quantitative data representing the average of two such studies. The abundance of cyclin E (CcnE, open circles, FIG. 5B) increased slowly during the first 8 hr after serum stimulation. Thereafter, the amount of cyclin E increased rapidly from 8-12 hr to 16 hr, increasing about five fold and remaining relatively constant for the duration of the study. CDK2 expression was more or less constant, increasing slightly between 16 hr and 24 hr after stimulation (triangles, FIG. 5B). Although both cyclin E and CDK2 remained relatively constant during early G1 (the first 8 hr after serum stimulation), the abundance of the cyclin E/CDK2 complex increased significantly within 4 hr after addition of serum (CcnE/CDK2, filled circles, FIG. 5B). Despite the rapid increase in cyclin E/CDK2 complexes during early G1 progression, there was little or no increase in cyclin E-dependent histone H1 kinase activity (E Kinase, filled squares, FIG. 5B) during the first 8 hr after serum stimulation. Table 4 summarizes some of these results.

TABLE 4
CELL CYCLE DISTRIBUTION OF
SERUM-STIMULATED 18LU CELLS

| | % Cells in | | % Cells in |
|---|---|---|---|
| Hrs | G1/G0 | % Cells in S | G2/M |
| 0 | 93.8(1.3) | 1.6(0.2) | 5.1(1.0) |
| 4 | 91.8(0.2) | 2.5(0.4) | 5.7(0.6) |
| 8 | 91.3(0.7) | 1.9(0.2) | 6.8(0.9) |
| 12 | 87.5(1.4) | 4.5(1.7) | 7.9(0.4) |
| 16 | 89.4(0.8) | 3.8(0.8) | 6.0(1.2) |
| 24 | 29.2(1.4) | 32.6(0.5) | 38.2(1.8) |

The data shown in FIG. 5B indicated that neither induction of cyclin E, nuclear uptake of CDK2, nor formation of cyclin E/CDK2 complexes is sufficient to account for the kinetics of activation of cyclin E/CDK2 kinase, although all three of these parameters are clearly important to kinase activation. For example, cyclin E-associated histone kinase activity (squares, FIG. 5B) did not begin to increase until about 8 hrs after serum stimulation, although cyclin E/CDK2 complexes (filled circles, FIG. 5B) had achieved near-maximum levels within this period of time. The delay in activation of cyclin E-dependent kinase, relative to formation of cyclin E/CDK2 complexes, suggests that kinase activity in early G1 may be constrained by a cyclin kinase inhibitor threshold.

### Example 19

### Subcellular Localization of CDK2, Cyclin E, Cip1, and Kip1

Immunocytochemical studies were carried out to determine if cyclin E and CDK2 share the same intracellular location in G0 cells.

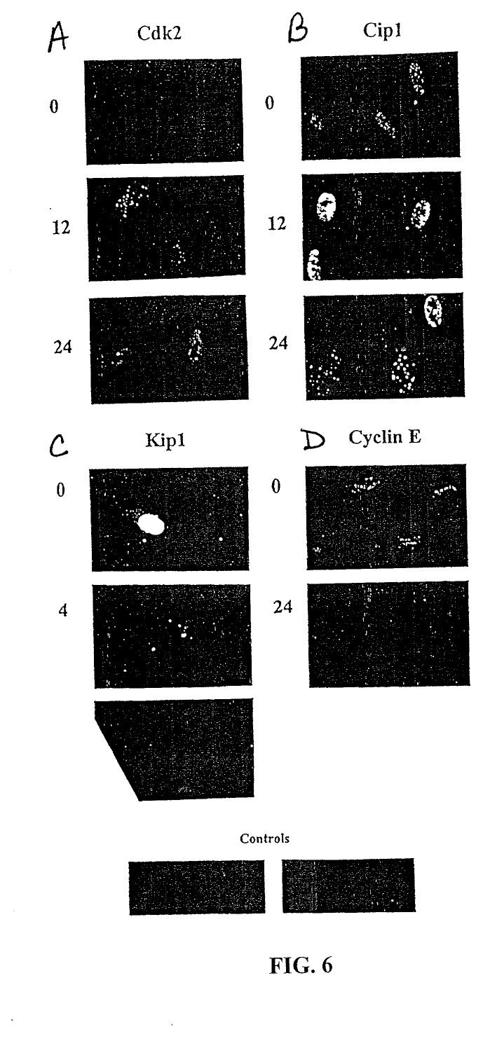

Serum-starved cells exhibited weak, diffuse cytoplasmic staining with CDK2 antibodies, but very little nuclear staining was observed (0 hr in FIG. 6A). Nuclear CDK2 staining increased after serum stimulation. Most of the nuclei stained for CDK2 within 12 hrs after addition of serum, although some nuclei were more intensely stained than others (as shown in FIG. 6A). The intranuclear distribution of CDK2 at 12 hrs after stimulation frequently gave rise to a punctate staining pattern, but the significance of this pattern is unknown. Nuclei ovserved 24 hrs after serum stimulation stained honogeneously and intensely for CDK2. Cyclin E was located primarily in the nuclei of quiescent cells, as shown in FIG. 6D; and the localization of cyclin E did not change after serum stimulation. These results are consistent with those reported for a transfected cell line that overexpresses cyclin E (Ohtsubo et al., 1995). The cyclin kinase inhibitors Cip1 and Kip1 were also localized primarily in the nuclei of quiescent cells. The staining intensity of Kip1 decreased rapidly after serum stimulation, suggesting a decrease in Kip1 expression. Both the abundance and the subcellular localization of Cip1 remained relatively constant after serum stimulation (FIG. 6B and FIG. 6C). However, within 24 hrs after addition of serum many of the Cip1-stained nuclei exhibited a pronounced punctate staining pattern, suggesting that the intranuclear localization of this inhibito may change during cell cycle progression. As indicated in FIG. 6B, about three-fourths of the cells exhibited this bright, punctate staining pattern at 24 hrs. One notes that about three-fourths of the cells in this culture had initiated S phase at this time (Table 4).

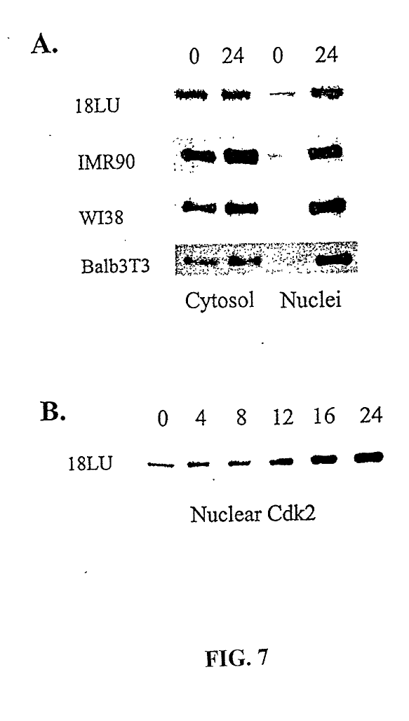

The data shown in FIG. 6A suggest that CDK2 is not abundant in nuclei of serum-starved cells. Alternatively, CDK2 could be bound to a nuclear factor that sequesters that epitope recognized by the antibodies used in these experiments. Subcellular fractionation was carried out to discriminate between these alternatives. Nuclear and cytosolic fractions were prepared from serum-starved cells and from cells that had been stimulated with serum for 24 hrs. The abundance of CDK2 in these fractions was measured by Western blotting, as shown in FIG. 7A. CDK2 in the cytosolic fractions increased by <2-fold after serum stimulation (FIG. 7A), consistent with the immunocytochemical data (FIG. 6A) which indicated that cytoplasmic CDK2 remains relatively constant after serum stimulation. Very little CDK2 was detected in the nuclear fractions from serum-starved LU cells, whereas the amount of nuclear CDK2 increased dramatically in serum-stimulated cells. Identical results were obtained with IMR90, WI38, and Balb3T3 fibroblasts (FIG. 7A).

The kinetics of nuclear accumulation are illustrated in FIG. 7B. There was a significant increase in nuclear CDK2 within 4 hrs after addition of serum to quiescent LU cells. The amount of nuclear CDK2 increased in a more or less linear fashion for 24 hrs after serum stimulation. The kinetics of nuclear accumulation of CDK2 paralleleg those of formation of the cyclin E/CDK2 complex, shown in FIG. 5B, at least for the first 16 hrs after serum stimulation.

Thus, although there is an abundance of cyclin E and CDK2 in G0 cells, the data illustrate that these two proteins do not reside in the same subcellular department. Consequently, quiescent cells contain very low concentrations of cyclin E/CDK2 complexes.

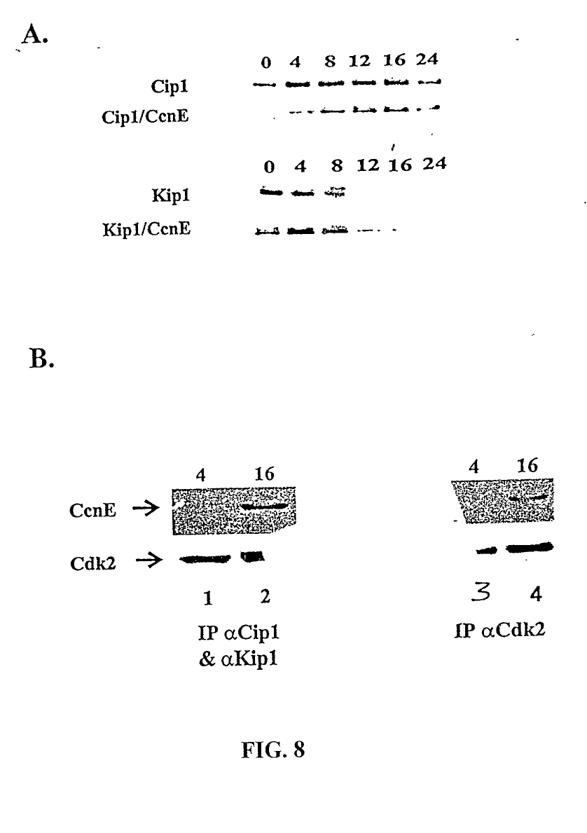

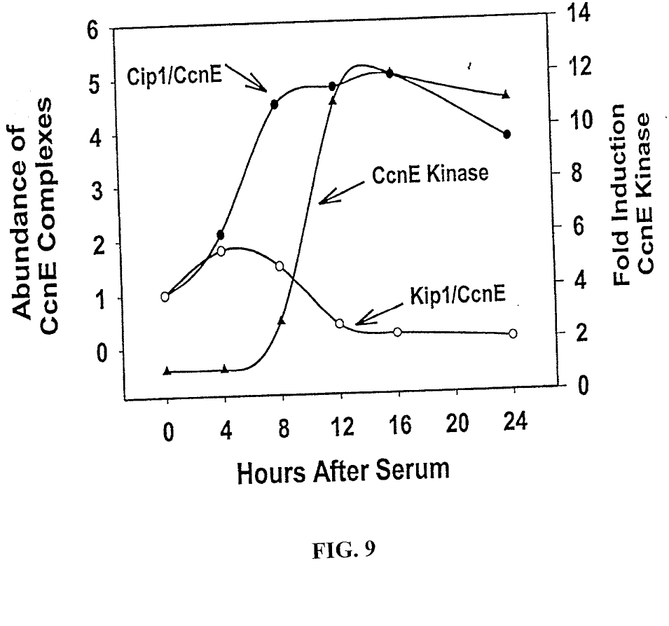

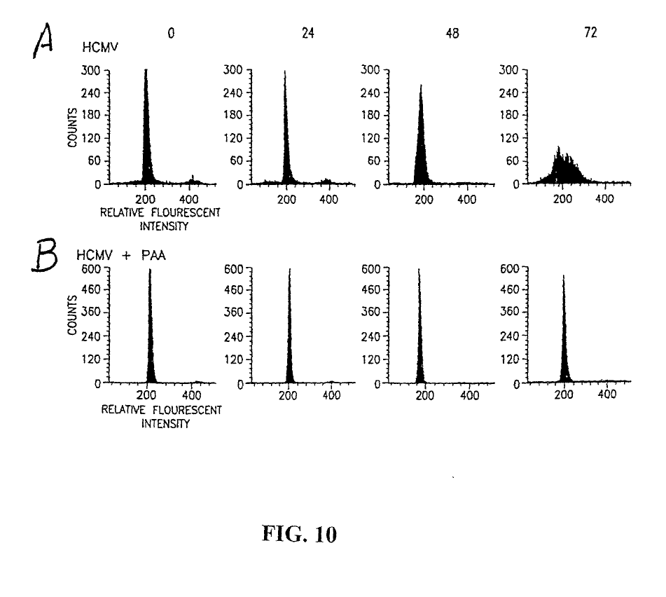

The abundance of the two principle CDK2 inhibitors, Cip1 and Kip1, was measured in serum-stimulated LU cells, as well as the formation of Cip1/cyclin E complexes and Kip1/cyclin E complexes, as shown in FIG. 8A and FIG. 8B. Serum stinulation caused a modest induction (about two-fold) of Cip1 (FIG. 8A). The binding of Cip1 to cyclin E (filled circles, FIG. 9) increased with kinetics that were notably different from those of Cip1 induction, but similar to the kinetics of formation of cyclin E/CDK2 complexes (FIG. 5B). The data are consistent with the conclusion that binding of Cip1 to cyclin E increased as the abundance of cyclin E/CDK2 complexes increased during the first few hrs after addition of serum. However, it appears that the binding capacity of Cip1 was exceeded within about 12 hrs, and there was little or no significant increase in the amount of Cip 1-containing complexes thereafter.

The abundance of Kip1 decreased rapidly after addition of serum to quiescent LU cells (FIG. 8A). This observation is consistent with the immunocytochemical data shown in FIG. 6C. It is significant that the amount of Kip1 bound to cyclin E increased during the first 8 hrs after stimulation, even though the total amount of Kip1 decreased during this time. This observation suggests that the binding capacity of Kip1 is in excess in G0 and during the first few hrs of G1. The amount of Kip1 bound to cyclin E began to fall after 8 hrs, and there was very little Kip1 bound to cyclin E 16 or 14 hrs after stimulation.

The data shown in FIG. 8A are consistent with the hypothesis that activation of cyclin E-dependent kinase during early G1 progression is attenuated by binding of CKIs to cyclin E/CDK2 complexes. This hypothesis predicts that all of most of the cyclin E/CDK2 should be bound to Cip1 or Kip1 at early time points (e.g., at 4 hrs after stimulation). On the other hand, there should be a significant amount of cyclin E/CDK2 that is free of inhibitors in late GI (e.g., 16 hrs after stimulation). An immunodepletion experiment was carried out to test these predictions. Extracts were prepared 4 hrs and 16 hrs after serum stimulation. The extracts were immunoprecipitated twice with a mixture of Cip1 and Kip1 antibodies, under conditions in which either antibody depletes >90% of its antigen in a single immunoprecipitation. The supernatant fractions were resolved by electrophoresis and assayed for cyclin E, as shown in lanes 1 and 2 of FIG. 8B. There was no detectable cyclin E in the CKI depleted supernatant fraction from 4 hour extracts (lane 1), although such supernatant factions contained CDK2. However, there was residual cyclin E that was not precipitated when CKI antibodies were added to 16 hour extracts (lane 2). In parallel, the supernatant fractions from CKI immunodepleted extracts (as shown in lanes 1 and 2) were subsequently precipitated with antibodies against CDK2, and the immunoprecipitates were assayed for cyclin E and Ckd2 (lanes 3 and 4). That fraction of cyclin E in 16 hour extracts that could not be precipitated with Cip1 and Kip1 antibodies (lane 2) could be precipitated with Cip1 and Kip1 antibodies, as shown in lane 4. The data in FIG. 8B indicate that, within the limits of detection, all of the cyclin E/CDK2 complexes in early G1 (4 hour) are saturated with either Cip1 or Kip1. However, the CKI binding capacity is not sufficient to saturate those cyclin E/CDK2 complexes that form in late G1 (16 hrs).

The quantitative data shown in FIG. 9 emphasize the temporal relationship between association of cyclin E with the two CKIs (Cip1/CcnE and Kip1/CcnE) and activation of cyclin E-dependent kinase (Ccn Kinase). The binding capacity of Cip1 saturated 8-12 hrs after stimulation, as the abundance of Kip1 fell (open circles). As a result, the threshold of Cip1 was exceeded while the Kip1 threshold decreased. Cyclin E-associated kinase activity accumulated rapidly at this time (triangles, FIG. 9).

### Example 20

### Cellular and Viral DNA Synthesis During Productive HCMV Infection

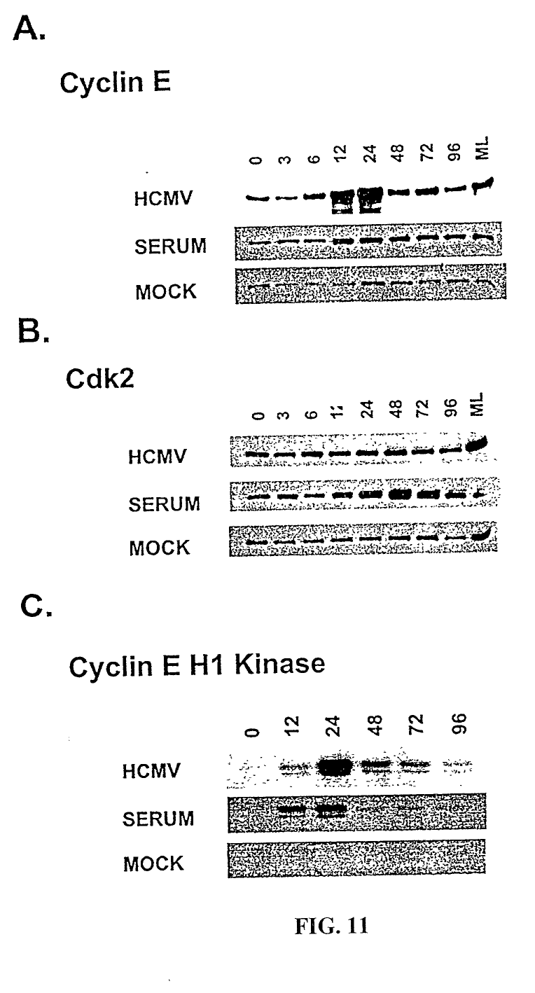

The DNA content of serum-arrested, HCMV-infected, subconfluent LU cells was analyzed by flow cytometry to determine to what extent productively infected cells initiate DNA synthesis. The results of a representative study are shown in FIG. 10A, and quantitative data are given in Table 5. Infected cells maintained a 2N DNA content for 24 hr after infection. Total DNA content of infected cells began to increase within 48 hr, and a substantial number of cells with >2N DNA content was observed 72 hr after infection. No increase in DNA content was observed in mock-infected cells. When confluent cultures were infected with HCMV, the results were essentially identical to those shown in FIG. 10A.

TABLE 5
Cell Cycle Distribution Following HCMV Infection

| | Percent Cells | Hrs After Treatment | | | |
|---|---|---|---|---|---|
| Treatment | in phase^{a} | 0 | 24 | 48 | 72 |
| HCMV^{b} | G0/G1 | 93.3 (1.2) | 91.9 (0.3) | 94.5 (2.2) | 74.8 (3.9) |
| | S | 1.6 (0.2) | 1.6 (0.6) | 3.8 (1.3) | 25.1 (3.9) |
| | G2/M | 5.1 (1.0) | 6.5 (0.3) | 1.8 (1.0) | 0 |
| HCMV^{b} + | G0/G1 | 93.3 (1.2) | 97.2 (0.4) | 97.1 (0.8) | 97.2 (0.3) |
| PAA^{c} | S | 1.6 (0.2) | 0.9 (0.1) | 1.5 (0.4) | 1.9 (0.3) |
| | G2/M | 5.1 (1.0) | 1.9 (0.3) | 1.3 (0.4) | 0.9 (0.1) |

^{a}The percent of cells in G0/G1, S, or G2/M of the cell cycle was determined following HCMV infection of subconfluent LU cells in the absence (HCMV) or presence of phosphonoacetic acid (HCMV + PAA). The data represent the average of three studies with standard deviation shown in parentheses.
^{b}5 PFU/cell
^{c}100 μg/ml

LU cells were productive for HCMV infection (Albrecht et. al., 1980a); so the increase in DNA content that one observed after HCMV infection was due to viral DNA replication. The relative contributions of viral and cellular DNA synthesis in productively infected cells was initially analyzed using phosphonoacetic acid (PAA), which, at a concentration of 100 μg/ml (0.75 mM), blocks viral DNA replication in human lung fibroblasts with little or no effect on cellular DNA synthesis or population doubling times of uninfected cultures (Huang, 1975). Expression of the viral late antigen pp28 requires replication of the HCMV genome (Depto and Stenberg, 1992; Re et. al., 1985; Meyer et. al., 1988), and pp28 expression could not be detected at any time between 24 and 96 hr after infection of LU cells in the presence of PAA. However, when cells were infected in the absence of PAA, antibodies against pp28 produced intense immunofluorescence beginning 24 hr after addition of the virus. The observation that PAA inhibited pp28 expression was consistent with the conclusion that the inhibitor blocked viral DNA replication. The specificity of PAA was confirmed by measuring cellular DNA content after serum stimulation of quiescent LU cells in the presence and absence of PAA. About 68% of serum-starved LU cells entered S, G2 or M phase within 24 hr after addition of serum in the absence of the inhibitor. When cells were stimulated with serum in the presence of PAA, about 57% of the cells were in S, G2, or M phase within 24 hr. These data indicated that, under the conditions employed in these studies, PAA was a specific inhibitor of viral DNA synthesis.

Next, the DNA content of LU cells as a function of time after HCMV infection of subconfluent cells in the presence of PAA was analyzed. As shown in FIG. 10B, there was no detectable increase in DNA content, under these circumstances. This observation indicated that the increase in total DNA content that was observed after viral infection depended upon viral DNA synthesis, and was consistent with the hypothesis that all or most of the DNA that was synthesized after viral infection was viral DNA.

The inhibitor studies shown in FIGS. 10A and 10B indicated that viral DNA synthesis was necessary for the increased DNA content that was observed in HCMV-infected LU cells.

### Example 21

### Activation of Cyclin E/CDK2 Kinase in HCMV-infected Cells

The effects of HCMV on G1 cyclins and CDKs were examined to measure aspects of cyclin/CDK activation in HCMV-infected LU cells. The effects of HCMV were examined on subconfluent, growth factor-deprived cells and density arrested cells. Subconfluent, growth factor-deprived cells are cells that are capable of undergoing G0→S phase progression after serum stimulation. Density arrested cells are incapable of initiating significant DNA synthesis after serum stimulation. Effects of the virus (HCMV) were contrasted with those of serum growth factors, which stimulate cell cycle progression by a mechanism that is partially understood (reviewed in Sherr, 1994; Draetta, 1994; Sherr, 1993).

The effect that HCMV-infection has on cyclin E was examined in subconfluent, serum-arrested LU cells. Cyclin E protein was induced within 12 hr after HCMV-infection (FIG. 11A). The effect of the virus was significantly more robust than that of serum growth factors. Cyclin E protein was induced >10-fold by HCMV and never more than 5-fold by serum. No significant induction of cyclin E in mock-infected cells was observed, demonstrating that the infection protocol does not result in serum-dependent mitotic stimulation, which might complicate interpretation of the results of viral infection.

Serum stimulation of subconfluent, serum-starved LU cells caused a 2-fold increase in CDK2 abundance (FIG. 11B); whereas neither the virus nor mock infection induced the catalytic partner of cyclin E. The activity of cyclin E/CDK2 kinase increased over 100-fold after HCMV infection, as evidenced by the ability of cyclin E immunoprecipitates to phosphorylate histone H1 (FIG. 11C). The effect of serum was less dramatic, although the activity of cyclin E-associated histone Hi kinase increased about 20-fold after serum stimulation of subconfluent cells (FIG. 11C). Mock infection had no effect on cyclin E/CDK2 activity.

### Example 22

### The Effect of HCMV on Expression of The Cip1 and Kip1

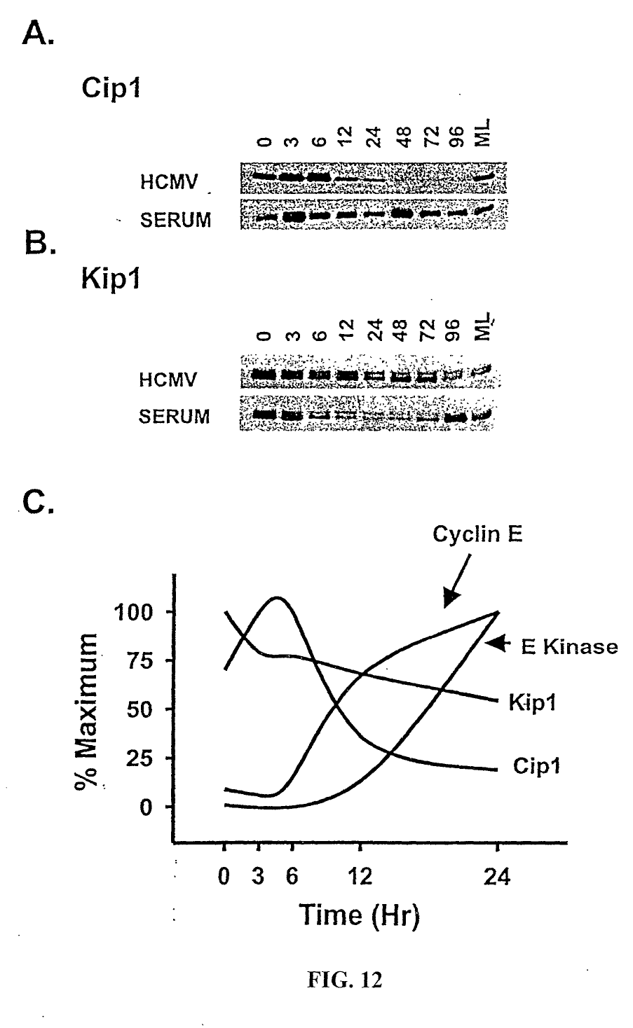

The abundance of Cip1 decreased rapidly after infection of subconfluent cells (FIG. 12A). Serum stimulation increased Cip1 expression (Li et. al., 1994; Nakanishi et. al., 1995). HCMV infection also inhibited expression of Kip1 (FIG. 12B), although the rate of inhibition was less rapid than that observed for Cip1. The kinetics of kinase activation lagged behind those of cyclin E induction, suggesting the existence of a cyclin kinase inhibitor threshold (FIG. 12C). This threshold was overcome about 12 hr after infection, as the expression of Cip1 decreased rapidly. Kip1 expression was reduced no more than 50% in 24 hr.

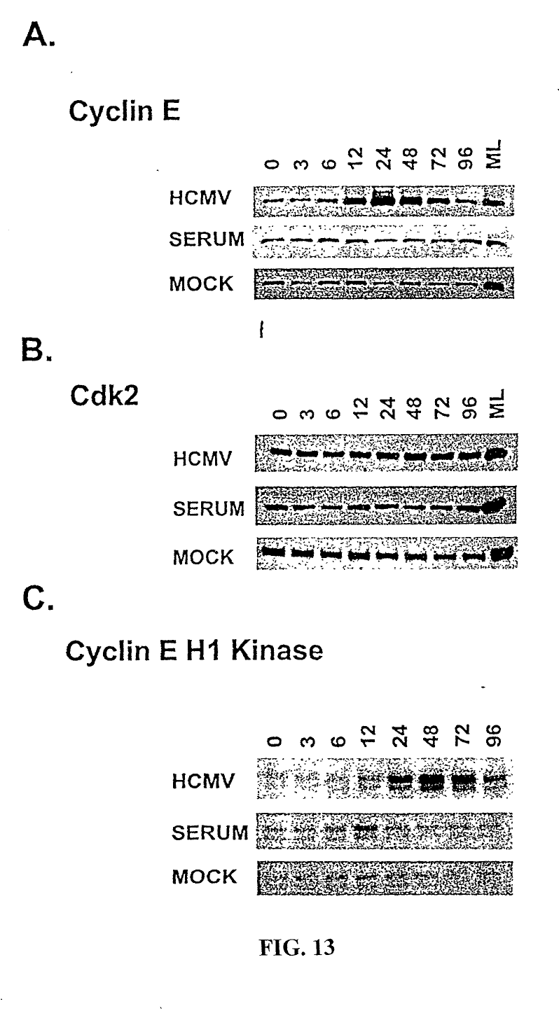

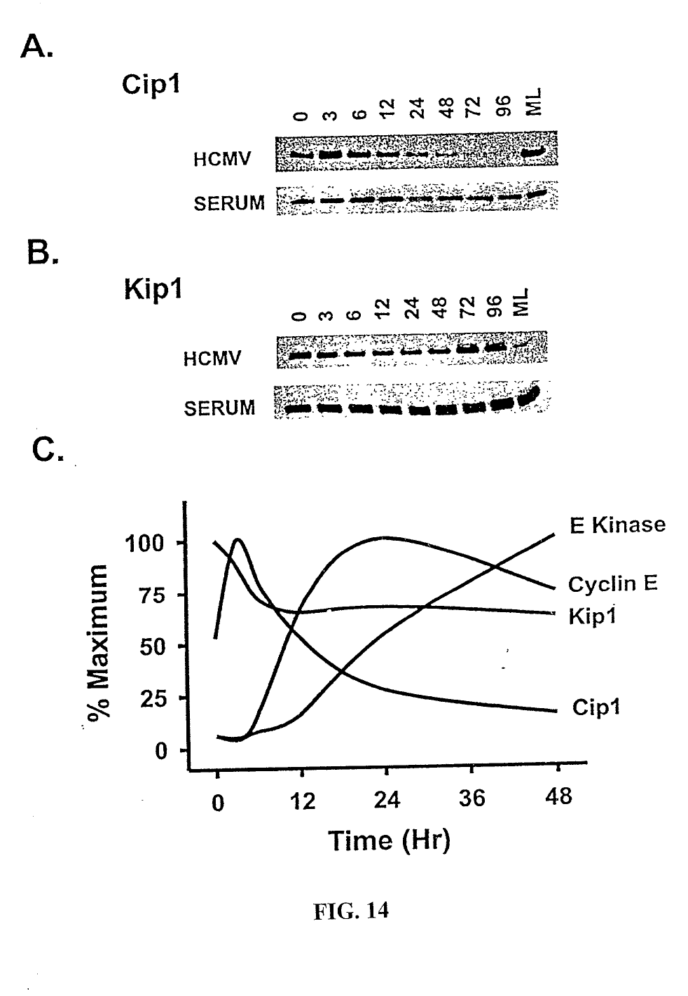

HCMV has in common with serum growth factors the ability to activate key GI progression factors in quiescent, subconfluent cells. However, the normal cellular targets of HCMV are not subconfluent cells. It is known that contact-arrested cells are recalcitrant to serum growth factor stimulation of G1 progression. As shown in FIG. 13A-FIG. 13C, HCMV induced cyclin E (FIG. 13A) and activated cyclin E-associated histone H1 kinase (FIG. 13C) with little or no effect on CDK2 expression (FIG. 13B). Serum had no significant effect on any of these parameters in contact-inhibited cells. Peak induction of cyclin E occurred 24 hr after infection of confluent cultures; whereas maximum activation of cyclin E/CDK2 kinase was observed at 48 hr (FIG. 13C). As was the case with subconfluent cells, activation of CDK2 attended inhibition of cyclin kinase inhibitor expression. Both Cip1 and Kip1 were inhibited in HCMV-infected cells (FIG. 14A and FIG. 14B). The kinetics of inhibition were somewhat slower than those observed in subconfluent cells (FIG. 14C), and closely paralleled activation of cyclin E-associated kinase activity. The data shown in FIG. 12C and FIG. 14C suggested that the kinetics of activation of cyclin E/CDK2 kinase were strongly influenced by inhibition of Cip1 in HCMV-infected cells. Inhibition of Kip1 expression may contribute to a lesser extent.

### Example 23

### HCMV Gene Expression and Cyclin E-dependent Kinase Activation

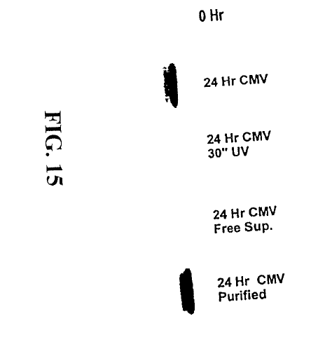

Initially, it was determined that UV irradiation of the virus blocked induction of cyclin E and activation of cyclin E/CDK2 (FIG. 15). The extent of irradiation was sufficient to inactivate detectable HCMV gene expression (Boldogh et. al., 1990). No immediate early gene expression was detected when cells were transfected with cyclin A (Schulze et. al., 1995). The observation that HCMV-infected fibroblasts neither initiate DNA synthesis nor induce cyclin A suggested that the virus might fail to induce the D-type cyclins. The abundance of cyclin D1 and its catalytic partner CDK4 were measured after HCMV infection of confluent cells. Cyclin D1 was not induced by the virus. Rather, the abundance of cyclin D1 decreased at about the time that cellular DNA synthesis stopped and viral DNA synthesis commenced. The expression of CDK4 did not change during the course of viral infection. Similar results were obtained with subconfluent cultures.

### Example 24

### Phosphorylation State of Retinoblastoma Gene

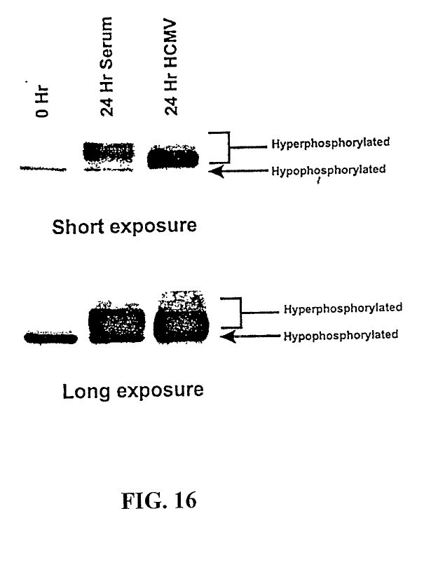

The product of retinoblastoma gene was examined following HCMV infection. LU cells were serum-arrested and then either stimulated with serum or infected with HCMV. After 24 hrs, the cells were harvested and cell lysates assayed for Rb expression by western blotting. Serum-arrested cells (0 hr) exhibited the hypophosphorylated form of Rb. Upon serum stimulation Rb became highly phosphorylated exhibiting at least three distinct phosphorylation states (FIG. 16). HCMV-infection also resulted in phosphorylation of Rb although the phosphorylation pattern differed from that observed for serum stimulation.

### Example 25

### HCMV is Capable of Causing CDK2 Translocation into the Nucleus

LU cells were grown on glass coverslips and arrested by serum-deprivation when they were 70-80% confluent. After 48 hr serum-deprivation the cells were either fixed for immunofluorescence, stimulated by addition of 20% FBS in EMEM, or infected with HCMV as described previously (Bresnahan et. al., 1996a). Infected cells were maintained in the reserved “spent” serum-free media to ensure that no stimulation would result from the presence of serum growth factors. The cells were fixed 24 hr after virus infection or serum stimulation. CDK2 antigen was detected by immunofluorescence using an anti-CDK2 antibody and a FITC-conjugated secondary antibody as previously described (Bresnahan et. al., 1996b).

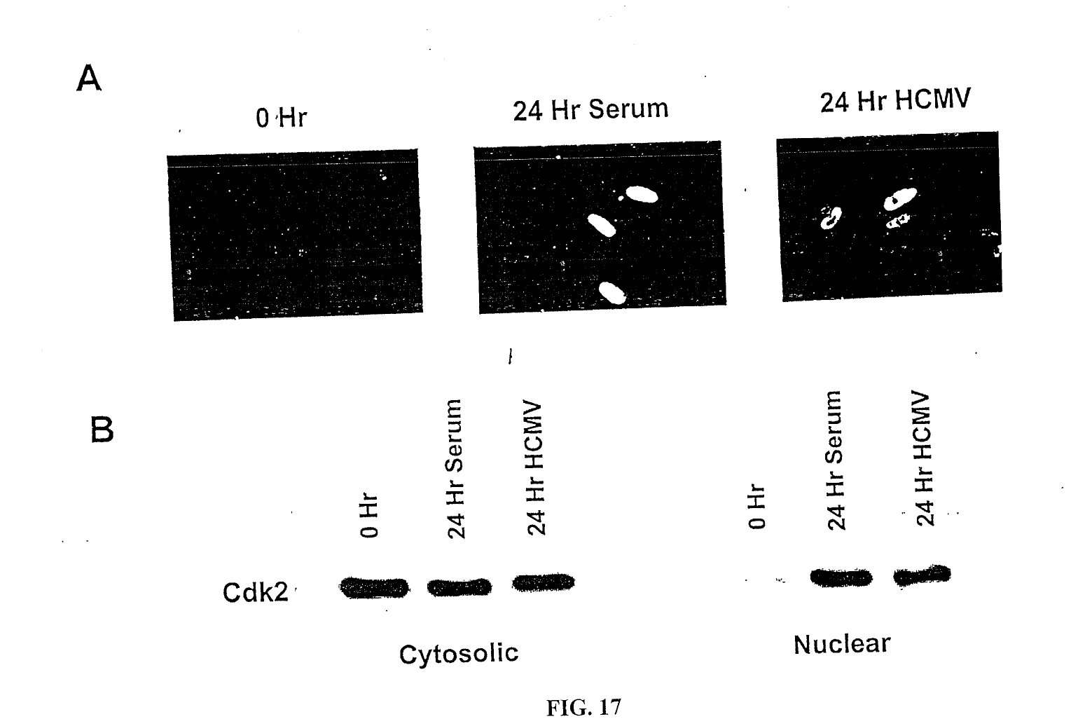

Cells arrested in G0 (0 hr) by serum deprivation exhibited a diffuse cytoplasmic immunofluorescence, with little or no nuclear staining. HCMV-infected or serum-stimulated cells exhibited a diffuse cytoplasmic staining pattern and intense nuclear immunofluorescence (FIG. 17A). These results suggest that HCMV, like serum, was capable of dramatically increasing the abundance of nuclear CDK2 within 24 hr post-infection, at which time cyclin E/CDK2 activity was maximal (FIG. 14C). Subcellular fractionation was carried out to confirm the immunocytochemical data. Nuclear and cytosolic fractions were prepared, as previously described (Bresnahan et. al., 1996b), from subconfluent, serum-arrested cells and from cells that had been HCMV-infected or serum-stimulated for 24 hr. The abundance of CDK2 present in these fractions were measured by western blotting as shown in FIG. 17B.

Very little CDK2 was detected in the nuclear fraction of serum-arrested cells (0 hr), whereas the amount of nuclear CDK2 increased dramatically in both HCMV-infected and serum-stimulated cells by 24 hr (FIG. 17B). These results confirmed the immunocytochemical data and demonstrated that HCMV (like serum) was capable of altering the subcellular localization of CDK2 in serum-arrested, subconfluent cells.

HCMV, but not serum, was also capable of activating cyclin E/CDK2 kinase in contact-inhibited cells (FIG. 13C). Next to determine if HCMV infection caused translocation of CDK2 into the nucleus, LU cells were cultured on glass coverslips and allowed to proliferate until the cells became confluent. The density-arrested cells were then fixed for immunofluorescence, infected with HCMV or stimulated with fresh EMEM containing 10% FBS, as described previously (Bresnahan et. al., 1996a). The cells were washed 24 hr later, and fixed for immunofluorescence as described above.

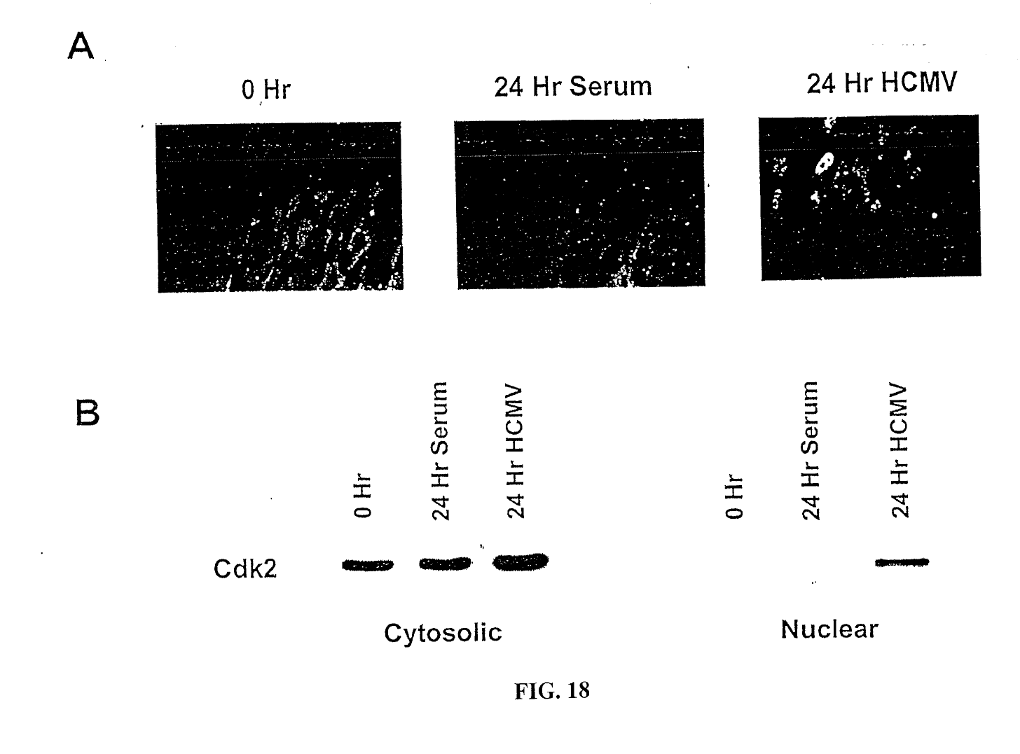

Cells arrested in G0 by contact inhibition demonstrated a diffuse cytoplasmic staining pattern with CDK2 antibodies. Little or no CDK2 was detected in the nuclei of these cells (FIG. 18A). Contact-inhibited cells that were HCMV-infected or treated with serum were also stained for CDK2. Cells treated with 10% FBS showed diffuse cytoplasmic staining with little or no nuclear staining, similar to that seen in untreated cells. However, cells infected with HCMV demonstrated an intense nuclear staining.

Subcellular fractionation was done to confirm the immunocytochemical data. Contact-arrested LU cells were infected with HCMV or treated with 10% FBS for 24 hr as described above. Nuclear and cytosolic fractions were prepared, and CDK2 abundance was determined by western blotting (FIG. 18B). In contact-arrested LU cells, CDK2 was predominantly located in the cytosolic fraction; and very little CDK2 was contained within the nuclear fraction. Similar results were obtained for contact-arrested cells that had been treated with 10% FBS for 24 hr. HCMV infection resulted in a large increase in the abundance of CDK2 in the nuclear fraction. These results confirmed the immunocytochemical data, showing that CDK2 is predominantly located within the cytoplasm of contact-arrested cells. However, HCMV infection, but not serum growth factors caused a dramatic increase in the abundance of CDK2 in the nuclei.

These findings suggested that the replication of HCMV depended upon the ability to activate cyclin E/CDK2 kinase activity.

### Example 26

### Activation of Cyclin E/CDK2 by HCMV

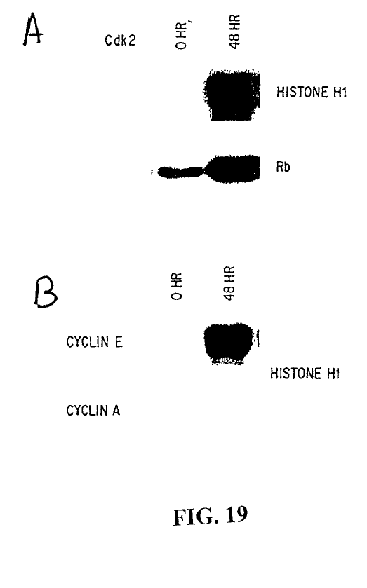

The data shown in FIGS. 19A and 19B illustrate HCMV's ability to activate CDK2. LU cells were arrested by contact-inhibition and then infected with HCMV. Cell lysates were prepared before infection (0 hr) and 48 hrs post-infection and assayed for CDK2 kinase activity. FIG. 19A shows that HCMV-infection resulted in a dramatic increase in CDK2 kinase activity using both histone HI and Rb as substrates. Kinase assays were also done on cyclin E and cyclin A immunoprecipitates from HCMV-infected cells. HCMV-infection resulted in an increase in cyclin E kinase activity with no induction of cyclin A kinase activity (FIG. 19B). These results demonstrated that the CDK2 activity that was induced in HCMV-infected cells was due to cyclin E/CDK2 complexes and not cyclin A/CDK2 complexes (Bresnahan et. al, 1996a).

### Example 27

### Inhibitation of HCMV Replication

An inhibitor of CDK2 activity, roscovitine and olomoucine, was used to determine that CDK2 activity was necessary for HCMV replication. The IC₅₀ for cyclin E/CDK2 inhibition in vitro by roscovitine is 0.71 μM (Meijer, 1996; Rudolph et. al., 1996).

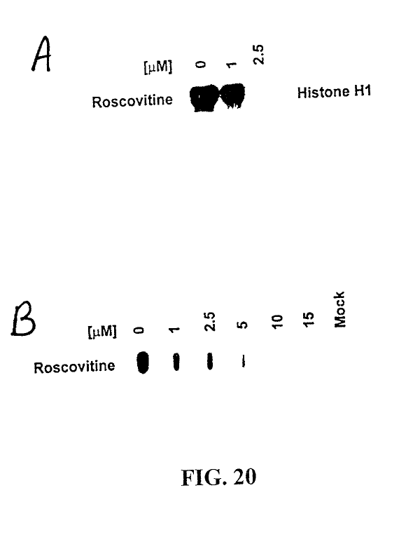

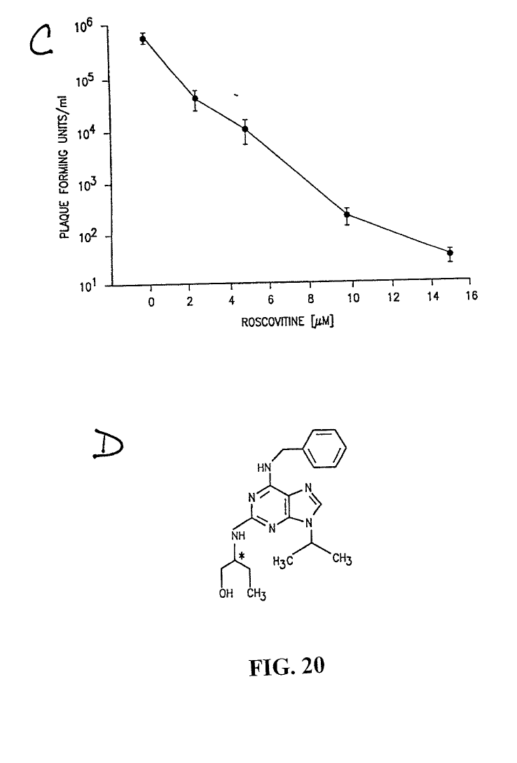

Density-arrested LU cells were infected for 1 hour after which the virus inoculum was removed and replaced with medium containing various concentrations of roscovitine. Total DNA was isolated from LU cells that had been infected for 72 hr, and the abundance of HCMV DNA was determined by slot blot hybridization. As FIG. 20B shows, HCMV DNA abundance was reduced by ˜50% in the presence of 1 μM roscovitine; and viral DNA abundance was reduced by >90% by 10 μM inhibitor. Consequently, roscovitine significantly reduced the production of infectious HCMV progeny, as shown in FIG. 20C. Infectious HCMV yields were reduced by >90% after addition of 2.5 μM roscovitine, and >99.9% inhibition occurred at 10 μM.

Since the IC₅₀ for roscovitine inhibition of CDK2 in vitro is 0.7 μM (Meijer, 1996; Rudolph et. al., 1996), the observation that both viral DNA synthesis and production of infectious virus particles was inhibited 50% at about 1 μM roscovitine suggested that inhibition of CDK2 accounts for inhibition of viral DNA replication. The chemical structure of roscovitine is shown in FIG. 10D.

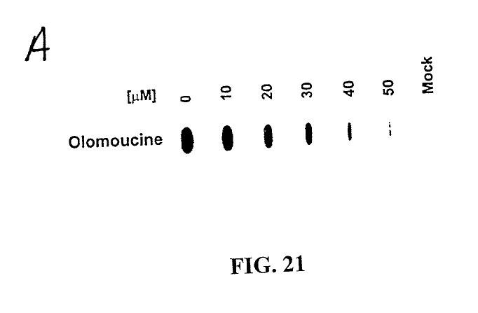

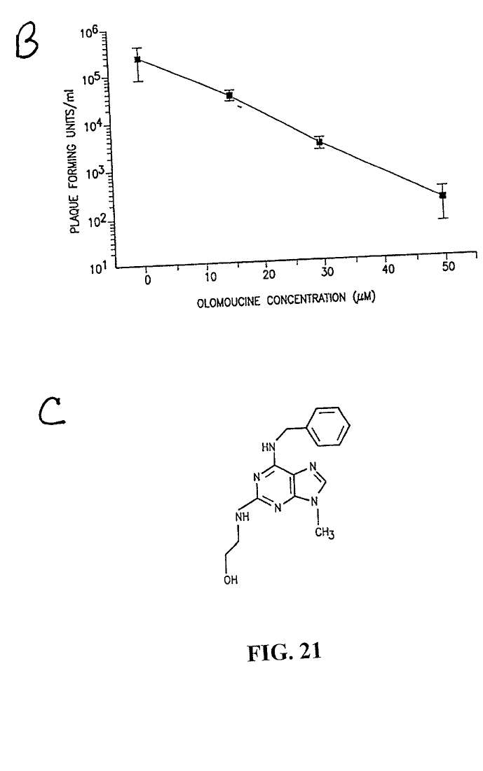

Similar results were obtained when olomoucine, a CDK2 inhibitor that is structurally related to roscovitine (Meijer, 1996), was used (FIGS. 21A-21C); however, the concentration of olomoucine that was required to inhibit viral DNA synthesis and virus yield was about 10-fold higher than the corresponding concentration of roscovitine. The IC₅₀ for olomoucine-mediated inhibition of CDK2 in vitro is 7 μM, (Vesely et. al., 1994) 10-fold higher than that of roscovitine. The chemical structure of olomoucine is shown in FIG. 21C.

### Example 28

### Other CDK Inhibitor Affect HCMV Replication

The observation that CDK activity becomes abnormally regulated in human cancers and the direct involvement of CDK5/p25 in Alzheimer's disease have stimulated the search for other chemical inhibitors of these kinases (Meijer, 1996; Meijer et. al., 1997; Garret et. al., 1999; Gray et. al., 1999 and Meijer et. al., 1999). All inhibitors identified so far act by competitive inhibition of ATP binding.

The search for CDK inhibitors has mostly been based on the use of CDK1/cyclin B as a molecular target. Starfish oocytes have become a widely used source of purified enzyme (Rialet et. al., 1991). Alternatively, recombinant CDKs have been expressed in insect cells and used to screen for inhibitors. The purified CDKs are assayed with 32P-y-ATP and an appropriate protein substrate such as histone H1 or the retinoblastoma protein in the presence of an increasing concentration of potential inhibitors. The dose-response curves provide IC50 values which are currently used to compare the efficiency of compounds to one another. Using these methods, eleven specific inhibitors have been identified (Table 6): olomoucine (Vesely et. al., 1994), roscovitine (Meijer et. al., 1997 and de Azevedo et. al., 1997), purvalanol (Gray et. al., 1998 and Chang et. al., 1999), CVT-313 (Brooks et. al., 1997), toyocamycin (Park et. al., 1996), flavopiridol (Sedlacek et. al., 1996), CGP60474 (Zimmermann, 1995), indirubin-3′-monoxime (Hoessel et. al., 1999), the paullones (Schultz et. al., 1999 and Zaharevitz et. al., 1999), γ-butyrolactone (Kitagawa et. al., 1993) and hymenialdisine (Meijer et. al., 2000). They all derive from structure/activity studies and from molecular modeling based on the crystal structure of the inhibitor in complex with CDK2. The chemical inhibitors have been characterized into six classes comprising: the purine-based compound olomoucine and its analogues, butyrolactone, flavopiridol, staurosporine and the related compound UCN-01, suramin and 9-hydroxyellipticine. Despite their chemical variety, these inhibitors all act by competing with ATP at the ATP-binding site of the catalytic subunit of the kinase.

TABLE 6

| | | CRYSTAL | |
|---|---|---|---|
| | IC₅₀ (μM) on | STRUCTURE | |
| INHIBITOR | CDK1/cyclin B | WITH CDK2 | SELECTIVITY |
| 6-Dimethylaminopurine | 120.000 | No | poor |
| Isopentenyladenine | 55.000 | [66] | poor |
| Olomoucine [18] | 7.000 | [66] | ++++ |
| Roscovitine [19, 20] | 0.450 | [20] | ++++ |
| CVT-313 [23] | 4.200 | No | unknown |
| Purvalanol A&B [21, 22] | 0.004 | [21] | ++++ |
| Flavopiridol [25] | 0.400 | [67] deschloroflavopiridol | ++ |
| Suramin | 4.000 | No | poor |
| 9-hydroxyellipticine | 1.000 | No | poor |
| Toyocamycin [24] | 0.880 | No | unknown |
| Staurosporine | 0.004 | [68] | poor |
| γ-Butyrolactone [30] | 0.600 | No | +++ |
| CGP60474 [26] | 0.020 | No | unknown |
| Kenpaullone [29] | 0.400 | No | ++++ |
| Alsterpaullone [28] | 0.035 | No | ++++ |
| Indirubin-3′-monoxime [27] | 0.180 | [27] | +++ |
| Hymenialdisine [31] | 0.022 | [31] | ++ |

The selectivity of some of these inhibitors is usually quite remarkable (Meijer, 1996; Meijer et. al., 1997; Garret et. al., 1999; Gray et. al., 1999 and Meijer et. al., 1999). Some inhibit CDK1, CDK2 and CDK5 but have no effect on CDK4 and CDK6. The structural reasons for such selectivity are unknown. No CDK4/CDK6 selective inhibitor has been reported yet. A few less selective inhibitors have been described (Meijer, 1996): 6-dimethylaminopurine, isopentenyladenine, suramin, staurosporine, UCN-01, 9-hydroxyellipticine. Although their use as tools in cell biology is limited, this is not necessarily the case in therapy. Furthermore they may constitute the basis for identification of more selective inhibitors as illustrated by olomoucine, roscovitine, purvalanol, which are all derived from the non-selective kinase inhibitors 6-dimethylaminopurine and isopentenyladenine.

In addition to chemical CDK inhibitors, peptides that mimic the CDK-inhibitory activity have been developed. Specifically, peptides corresponding to p16 or p21 have been generated with differing cellular effects.

Similar results were obtained using several CDK inhibitors. Density-arrested LU cells were infected for 1 hour after which the virus inoculum was removed and replaced with medium containing various concentrations of CDK inhibitors. Infectious virus was harvested 96 hrs later and assayed for infectivity by plaque assay. Table 7 illustrates data obtained using other CDK inhibitors to inhibit HCMV replication.

TABLE 7
Inhibition of Human Cytomegalovirus Infectious Yields by
Inhibitors of Cyclin-dependent Kinase Activity

| | Concentration | Percent |
|---|---|---|
| Inhibitor | (μM) | inhibition |
| NG97 (aminopurvalanol | 100 | 99.9992 |
| | 33 | 99.9992 |
| | 10 | 99.9984 |
| | 3.3 | 99.94 |
| | 10 | 51.33 |
| Roscovitine | 100 | >99.9984 |
| | 33 | 99.996 |
| | 10 | 96.47 |
| | 3.3 | 90.67 |
| | 10 | 91.33 |
| Indirubin-3′-monoxime | 100 | 99.9992 |
| | 33 | 99.9976 |
| | 10 | 99.9988 |
| | 3.3 | 56.69 |
| | 10 | 26.67 |
| RP107 | 100 | 99.9984 |
| | 33 | 99.9968 |
| | 10 | 99.98 |
| | 3.3 | 96.67 |
| | 10 | 74.67 |
| Alsterpaullone | 100 | 99.994 |
| | 33 | 99.998 |
| | 10 | 99.997 |
| | 3.3 | 99.998 |
| | 10 | 99.998 |
| Flavopiridol | 100 | 99.998 |
| | 33 | 99.998 |
| | 10 | 99.998 |
| | 3.3 | 99.998 |
| | 10 | 99.998 |

Percent inhibition calculated as PFUc − PFUi/PFUc; where PFUc = plaque forming units/ml for mock-treated cells, PFUi = plaque forming units/ml for cells treated with drug at the indicated concentration.

### Example 29

### CDK Inhibitors on Non-infected Cells

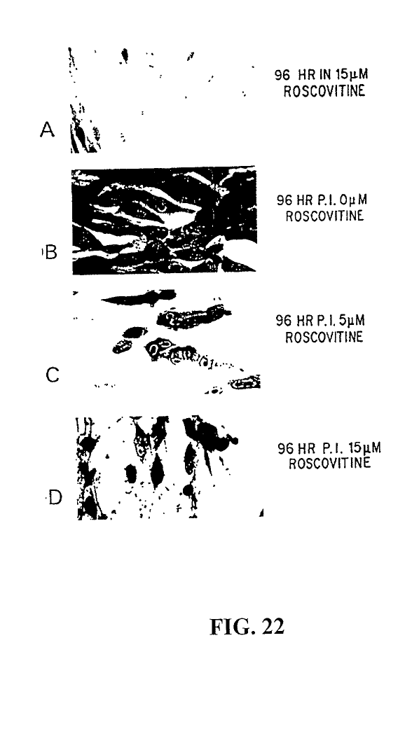

Hematoxylin and eosin staining was also done on non-infected and infected cells both in the absence and presence of roscovitine. Non-infected cells were treated with 15 μM roscovitine for 96 hr and subsequently stained with hematoxylin and eosin. FIG. 22A demonstrates that non-infected LU cells treated with roscovitine show no morphological changes. Infected cells were also stained 96 hr post-infection and examined for morphological changes. As FIG. 22B shows, infected, untreated cells were flat in shape and displayed large nuclear inclusions characteristic of HCMV infection. In the presence of 5 μM roscovitine the infected cells were rounded and appeared to be undergoing cell death. However, even in the presence of 5 μM roscovitine small nuclear inclusions are evident indicating that the cells are infected (FIG. 22C). Cells infected and treated with 15 μM roscovitine were clearly dying or dead by 96 hr after infection (FIG. 22D). Similar morphological changes were observed for cells infected and treated with olomoucine. These results suggest that HCMV-infected cells treated with inhibitors of CDK2 activity undergo cell death that does not result from treatment with inhibitor alone.

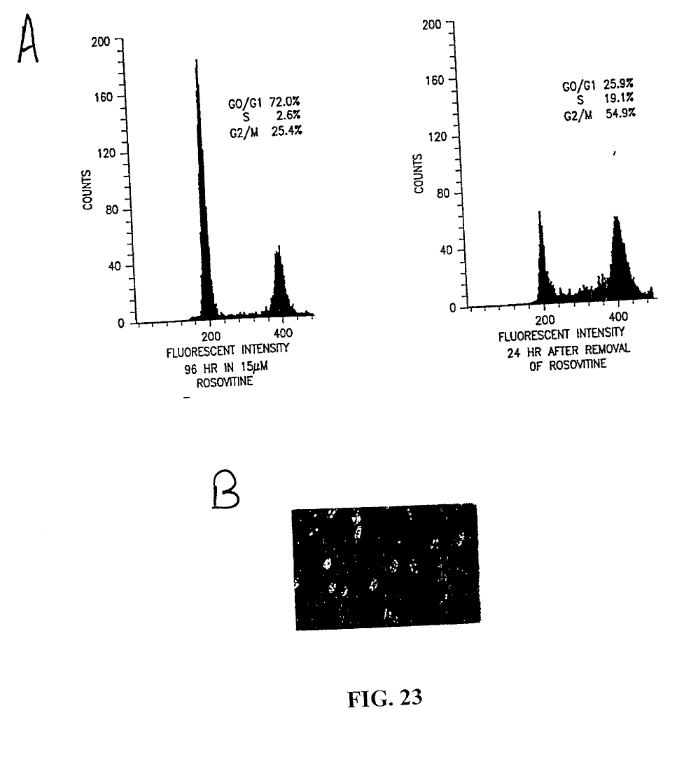

To determine whether drug-associated cellular toxicity was responsible for the reduced HCMV replication, the effects of both CDK inhibitors (e.g., roscovitine and olomoucine) on non-infected cells were investigated. Non-infected cells treated with 15 μM roscovitine for 96 hr were arrested in G0/G1 or G2/M phase (FIG. 23A) and did not present with any obvious morphological changes (FIG. 23A). In addition, more than 70% of the cells that had been exposed to roscovitine or olomoucine in this fashion were able to incorporate bromodeoxyuridine (BrdU) within 24 hr after removal of the inhibitor (FIG. 23B). Similar results were obtained when roscovitine- or olomoucine-treated cells were analyzed for cell cycle progression (using flow cytometry) after removal of the drug (FIG. 23A).

### Example 30

### Dominant Negative CDK2 Mutant Inhabits CDK2 Activity

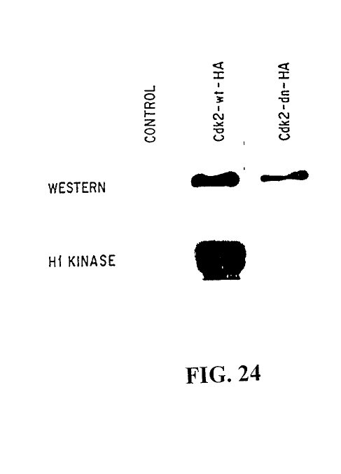

A previously characterized dominant negative CDK2 mutant (van den Heuval and Harlow, 1993) was used to show that the effects of roscovitine on HCMV replication are due to inhibition of CDK2. U-373 cells were used in these studies because of the low efficiency of transfection of LU cells. U-373 cells were transiently transfected with expression vectors encoding hemagglutinin (HA)-tagged wild-type Cdk2 (pCMVCdk2-wt-HA) or HA-tagged dominant negative Cdk2 (pCMVCdk2-dn-HA). The hemagglutinin tag allowed the inventors to distinguish between endogenous and exogenous Cdk2 by the use of specific hemagglutinin antibodies. Cells were harvested 48 hrs after transfection and assayed for Cdk2-wt-HA and Cdk2-dn-HA expression and kinase activity. Western blotting with HA antibody showed that cells transfected with either wild-type or dominant negative Cdk2 expressed the exogenous protein (FIG. 24). Histone H1 kinase activity was associated with CDK2 wild-type HA immunoprecipitates but not with the CDK2 dominant negative HA precipitates (FIG. 24).

### Example 31

### HCMV Replication and CDK2 Activity

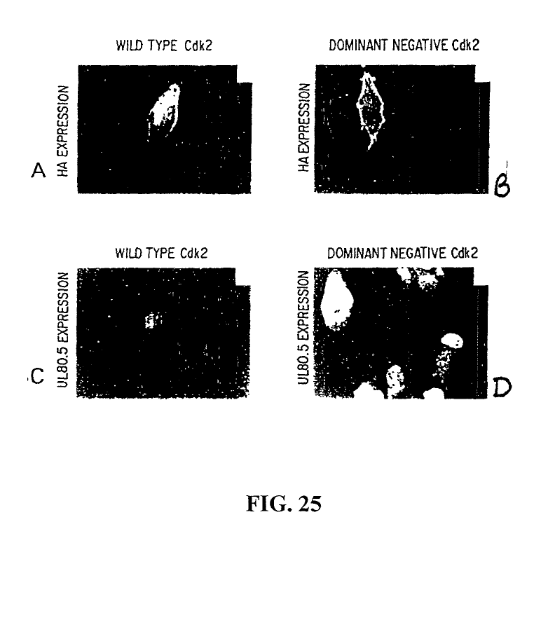

To determine if CDK2 activity is required for HCMV replication, dual immunofluorescent staining was used to assay for expression of the HA-tagged CDKs and for the HCMV late antigens encoded by UL80.5, in transfected U-373 cells that were also infected with HCMV. As shown in FIGS. 15A-15D, cells that expressed the dominant negative CDK2 mutant (shown in FIG. 25B) did not express UL80.5 late antigens (FIG. 25D). Cells that expressed wild-type Cdk2 (FIG. 25A) supported viral replication, as evidenced by expression of the UL80.5 late gene products (FIG. 25C). Three independent studies of this kind were done; and the percentage of infected cells, cells expressing Cdk2-wt-HA plus UL80.5, or cells expressing Cdk2-dn-HA plus UL80.5 were determined. Transfection efficiencies were similar for both wild type and dominant negative CDK2. About 33% of the cells in the infected cultures expressed UL80.5 late antigens. This efficiency of infection was observed irrespective of whether the cells were transfected with wild type or dominant negative derivatives of CDK2. The susceptibility of U-373 cells to HCMV was consistent with published data (Ripalti et. al., 1995). The efficiency of transient expression of Cdk2 derivatives is much lower than the frequency of virus infection. It was estimated that <5% of the U-373 cells, and therefore <5% of the infected cells, expressed either HA-tagged CDK2 derivatives. Similar results were obtained with β-galactosidase expression vectors. Nevertheless, as Table 8 shows, about 37% of the cells that expressed the wild type HA-tagged CDK2 derivative also expressed UL80.5 late antigens. This observation demonstrated that transient expression of HA-tagged wild type Cdk2 derivative has no significant effect on viral replication, as assessed by expression of viral late antigen. On the other hand, only 2% of cells that expressed the dominant negative Cdk2 mutant also expressed UL80.5 late antigens (p<0.0001). This observation indicated that CDK2 activity is vital for HCMV replication and inhibition of CDK2 is sufficient to inhibit viral replication.

Table 8 shows the percent of cells expressing CDK2-HA and HCMV UL80.5 antigens. U-373 cells were transiently transfected with HA-tagged wild type or dominant negative Cdk2 and subsequently infected with HCMV. The percent of infected cells was determined by measuring expression of HCMV UL80.5 antigens. Multiple random fields were counted to accumulate about 150 total cells, of which about one-third expressed UL80.5. The percent of cells expressing UL80.5 antigens was also determined from cells expressing HA-tagged wild type or dominant negative CDK2. In this case, multiple fields were counted to accumulate about 150 HA-positive cells, which were scored for expression of UL80.5. Statistical significance was estimated by Student's t-test, comparing infected cells expressing HA to the percent infected cells in the cultures.

TABLE 8
Percent of Cells Expressing CDK2-HA
and HCMV UL80.5 Antigens

| Cells Expressing | Study 1 | Study 2 | Study 3 | Mean % (±s.d.) |
|---|---|---|---|---|
| UL80.5 Antigens | 52/156 | 46/156 | 53/150 | 33 (±2) |
| | 33% | 30% | 35% | |
| PCMVCDK2-wt-HA + | 58/148 | 56/154 | 53/147 | 37 (±1) |
| UL80.5 Antigens | 39% | 37% | 36% | p > 0.05 |
| PCMVCDK2-dn-HA + | 3/151 | 5/161 | 4/159 | 2.6 (±0.6) |
| UL80.5 Antigens | 2% | 3% | 3% | p < 0.0001 |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Albrecht, T., Boldogh, I., Fons, M., Lee, C. H., AbuBakar, S., Russell, J. M. and Au, W. W. 1989. Subcell. Biochem. 15:157-202.

Albrecht, T., Boldogh, I., Fons, M. P. and Deng, C. Z. 1992. J. Invest. Dermatol., 98:29S-35S.

Albrecht, T., Knutson, E., and Chen, Z. 1999. Abstracts of the FASEB Summer Research Conference #1162 (Calpains), p. 34.

Albrecht, T., Lee, C. H., Speelman, D. J. and Steinsland, O. S. 1987. Proc. Soc. Exp. Biol. Med. 186:41-46.

Bibb J A, Snyder G L, Nishi A, Yan Z, Meijer L, Fienberg A A, Tsai L H, Kwon Y T, Girault J-A, Czernik A J, Huganir R L, Hemmings H C, Nairn A C, Greengard P (1999) Nature 402: 669-671

Bresnahan, W., Boldogh, I., Chi, P., Thompson, E. A. and Albrecht, T. 1997a. Virology, 231:239-247.

Bresnahan, W. A., Albrecht, T., and Thompson, A. 1998. J. Biol. Chem. 273:22075-22082.

Bresnahan, W. A., Thompson, E. A., and Albrecht, T. 1997b. J. Gen. Virol., 78:1993-1997.

Brooks E E, Gray N S, Joly A, Kerwar S S, Lum R, Mackman R L, Norman T C, Rosete J, Rowe M, Schow S R, Schultz P G, Wang X, Wick M M, Shiffman D (1997). J Biol Chem 272: 29207-29211

Chang Y T, Gray N G, Rosania G R, Sutherlin D P, Kwon S, Norman T C, Sarohia R, Leost M, Meijer L, Schultz P G (1999). Chem & Biol 6: 361-375

Chen, Z., and Albrecht, T. 1998.

de Azevedo W F, Leclerc S, Meijer L, Havlicek L, Strnad M, Kim S-H (1997). Eur J Biochem 243: 518-26

Garrett M D, Fattaey A (1999). Curr Opin Genet & Dev 9: 104-111

Gray N, Detivaud L, Doerig C, Meijer L (1999). Curr Medicin Chem 6, 859-876

Gray N, Wodicka L, Thunnissen A M, Norman T, Kwon S, Espinoza F H, Morgan D O, Barnes G, Leclerc S, Meijer L, Kim S H, Lockhart D J, Schultze P (1998). Science 281: 533-538

Guo M, Hay B A (1999). Curr Opin Cell Biol 11: 745-752

Hoessel R, Leclerc S, Endicott J, Noble M, Lawrie A, Tunnah P, Leost M, Damiens E, Marie D, Marko D, Niederberger E, Tang W, Eisenbrand G, Meijer L (1999) Nature Cell Biol 1, 60-67

Kitagawa M, Okabe T, Ogino H, Matsumoto H, Suzuki-Takahashi I, Kokubo T, Higashi H, Saitoh S, Taya Y, Yasuda H, Ohba Y, Nishimura S, Tanaka N, Okuyama A (1993) Oncogene 8: 2425-2432

Lee M G, Nurse P (1987). Nature 237: 31-35

Meijer L (1996). Trends in Cell Biol 6: 393-397

Meijer L, Borgne A, Mulner O, Chong J P J, Blow J J, Inagaki N, Inagaki M, Delcros J G, Moulinoux J P (1997) Eur J Biochem 243: 527-36

Meijer L, Guidet S, Philippe M (editors) (1997) Progress in Cell Cycle Research, vol.3, Plenum Press, New York, 321 pp (24 chapters)

Meijer L, Jezequel A Ducommun B (editors) (2000). Progress in Cell Cycle Research, vol. 4, Plenum Press, New York, 248 pp (21 chapters)

Meijer L, Kim S H (1997). Methods in Enzymology, “Cell Cycle Control”, vol. 283: 113-128

Meijer L, Leclerc S, Leost M (1999) Pharmacol & Ther 82: 279-284

Meijer L, Thunissen A M W H, White A, Gamier M, Nikolic M, Tsai L H, Walter J, Cleverley K E, Salinas P C, Wu Y Z, Biernat J, Mandelkow E M, Kim S-H, Pettit G R (2000). Chem & Biol 7: 51-63

Meyerson M, Enders G H, Wu C L, Su L K, Gorka C, Nelson C, Harlow E, Tsai L H (1992). EMBO J 11: 2909-2917

Morgan D (1997). Annu Rev Cell Dev Biol 13: 261-291

Nurse P, Bissett Y (1981). Nature 292: 558-60

Park S G, Cheon J Y, Lee Y H, Park J-S, Lee K Y, Lee C H, Lee S K (1996) A specific inhibitor of cyclin-dependent protein kinases, CDC2 and CDK2. Mol Cells 6: 679-83

Patrick G N, Zukerberg L, Nikolic M, De la Monte S, Dikkes P, Tsai L H (1999). Nature 402: 615-622

Plowman G D, Sudarsanam S, Bingham J, Whyte D and Hunter T (1999). Proc Natl Acad Sci USA 96: 13603-13610

Rialet V, Meijer L (1991). Anticancer Res 11: 1581-1590

Schang, L. M., Bantly, A., Rosenberg, A., and Schaffer, P. A. 2000. Antiviral Res. 46:A73.

Schultz C, Link A, Leost M, Zaharevitz D W, Gussio R, Sausville E A, Meijer L, Kunick C (1999). J Med Chem 42: 2909-2919

Sedlacek H H, Czech J, Naik R, Kaur G, Worland P, Losiewicz M, Parker B, Carlson B, Smith A, Senderowicz A, Sausville E (1996). Internat J Oncol 9: 1143-1168

Vesely J, Havlicek L, Strnad M, Blow J J, Donella-Deana A, Pinna L, Letham D S, Kato J Y, Detivaud L, Leclerc S, Meijer L (1994). Eur J Biochem 224: 771-86

Vogt P K, Reed S I (1998). Current Topics in Microbiology and Immunology, Springer Verlag, 169 pp

Zaharevitz D, Gussio R, Leost M, Senderowicz A M, Lahusen T, Kunick C, Meijer, L, Sausville E A (1999). Cancer Res 59: 2566-2569

Zimmermann J (1995). PCT Ciba-Geigy, WO 95/09853

What is claimed is:

1. A method of treating an organism infected or suspected of being infected by a virus, bacterium, or parasite comprising administering a cyclin-dependent kinase inhibitor to the organism.

2. The method of claim 1, wherein the organism is a human.

3. The method of claim 1, wherein administering is parenteral.

4. The method of claim 1, wherein administering is alimentary.

5. The method of claim 1, wherein administering is topical.

6. The method of claim 1, wherein administering is inhalation.

7. The method of claim 1, wherein the organism is infected or suspected of being infected by a DNA virus.

8. The method of claim 7, wherein the DNA virus is a parvovirus, papovavirus, hepadnavirus, adenovirus, herpesvirus or poxvirus.

9. The method of claim 1, wherein the inhibitor is administered in a therapeutically effective amount to inhibit DNA replication.

10. The method of claim 9, wherein the therapeutically effective amount is from about 0.1 μg/kg to about 1000 μg/kg.

11. The method of claim 1, wherein the inhibitor is administered in a prophylactically effective amount to inhibit DNA replication.

12. The method of claim 11, wherein the prophylactically effective amount is from about 0.1 μg/kg to about 1000 μg/kg.

13. The method of claim 1, further comprising administering a second agent that is capable of inhibiting the virus, bacterium, or parasite.

14. The method of claim 1, further comprising administering an antiviral agent.

15. A method of screening for a modulator of cyclin-dependent kinase comprising:
obtaining a cyclin-dependent kinase;
contacting the cyclin-dependent kinase with a candidate substance; and
assaying for cyclin-dependent kinase activity.

16. The method of claim 15, wherein cyclin-dependent kinase is CDK1, CDK2, CDK3, CDK4, CDK5, CDK6, CD7, CDK8 or CDK9.

17. The method of claim 15, further defined as comprising determining whether the candidate substance inhibits the cyclin-dependent kinase.

18. The method of claim 15, further defined as comprising determining whether the candidate substance competitively inhibits ATP binding.

19. The method of claim 15, wherein the candidate substance is 6-dimethylaminopurine, isopentenyladenine, olomoucine, roscovitine, CVT-313, purvalanol A&B, flavopiridol, suramin, 9-hydroxyellipticine, toyocamycin, staurosporine, γ-butyrolactone, CGP60474, kenpaullone, alsterpaullone, indirubin-3′-monoxime or hymenialdisine.

20. The method of claim 15, wherein obtaining the cyclin-dependent kinase protein comprises procuring an expressed cyclin-dependent kinase protein.

21. The method of claim 20, wherein cyclin-dependent kinase protein is procured by isolation from a cell.

22. The method of claim 15, wherein contacting the cyclin-dependent kinase protein with the substance is performed in a cell free system.

23. The method of claim 15, wherein contacting the cyclin-dependent kinase protein with the substance is performed in a cell.

24. The method of claim 15, wherein contacting the cyclin-dependent kinase protein with the substance is performed in vivo.

25. The method of claim 15, wherein the method further comprises modifying a substance to create the candidate substance.

26. A method of screening a candidate substance for cyclin-dependent kinase binding activity comprising:
providing a cyclin-dependent kinase protein;
contacting the cyclin-dependent kinase protein with the candidate substance; and
determining whether the candidate substance binds to the cyclin-dependent kinase protein.

27. The method of claim 26, further defined as comprising determining whether the candidate substance binds to the ATP-binding site of the catalytic subunit of cyclin-dependent kinase.

28. The method of claim 27, wherein the candidate substance is 6-dimethylaminopurine, isopentenyladenine, olomoucine, roscovitine, CVT-313, purvalanol A&B, flavopiridol, suramin, 9-hydroxyellipticine, toyocamycin, staurosporine, γ-butyrolactone, CGP60474, kenpaullone, alsterpaullone, indirubin-3′-monoxime or hymenialdisine.

29. The method of claim 26, further defined as comprising determining whether the candidate substance inhibits ATP binding of cyclin-dependent kinase.

30. The method of claim 26, wherein contacting the cyclin-dependent kinase protein with the candidate substance is performed in a cell free system.

31. The method of claim 26, wherein contacting the cyclin-dependent kinase protein with the candidate substance is performed in a cell.

32. The method of claim 26, wherein contacting the cyclin-dependent kinase protein with the substance is performed in vivo.

33. A method of screening putative inhibitors of virus, bacterial, or parasite replication comprising:
contacting a cell with a virus, bacteria, or parasite;
contacting the cell with an inhibitor of cyclin-dependent kinase;
measuring a cellular response; and
measuring any yield of virus, bacteria, or parasite.

34. The method of claim 33, wherein the cellular response is phospholipase C activity, phospholipase A2 activity, phospholipid mobilization and metabolism, protein kinase C activity, Ca²⁺ fluctuations, other ion fluctuations, protein kinase activities, cAMP, cGMP, activation of DNA binding proteins, transcription of cellular genes or modification of the cytoskeleton or adhesion apparatus.

35. The method of claim 33, wherein the screening method is performed in vitro.

36. The method of claim 33, wherein the screening method is performed in vivo.

37. The method of claim 33, wherein the cell is contacted with a DNA virus.

38. The method of claim 37, wherein the DNA virus is a parvovirus, papovavirus, hepadnavirus, adenovirus, herpesvirus or poxvirus.

39. The method of claim 33, wherein the inhibitor inhibits cyclic-dependent kinase ATP binding.
